(19) United States
(12) Patent Application Publication
(10) Pub. No.: US 2019/0277856 A1
(43) Pub. Date: Sep. 12, 2019

(54) METHODS FOR ASSESSING RISK OF INCREASED TIME-TO-FIRST-CONCEPTION

(71) Applicant: Celmatix Inc., New York, NY (US)

(72) Inventors: Piraye Yurttas Beim, New York, NY (US); Genevieve Galarneau, Jersey City, NJ (US)

(21) Appl. No.: 16/287,607

(22) Filed: Feb. 27, 2019

Related U.S. Application Data
(60) Provisional application No. 62/636,391, filed on Feb. 28, 2018.

(51) Int. Cl. G01N 33/68; G16B 25/30; G16B 20/00; G16H 50/30; G16H 20/10; G06F 17/18; C12Q 1/68

(52) U.S. Cl. CPC G01N 33/689; G16B 25/30; G16B 20/00; C12Q 1/68; G16H 20/10; G06F 17/18; G16H 50/30

(57) ABSTRACT

Genetic factors that regulate uterine development, endometrial function, and gonadotropin signaling are associated with increased time to conception. Newly discovered loci are shown to have a highly significant correlation with the phenotype. These newly discovered associations form the basis for methods of diagnosing and treating infertility. These loci are useful alone or in combination with other biomarkers and phenotypic markers to diagnose or assess the risk of an increased time to conception phenotype, and can help guide diagnosis and treatment while improving outcomes.

### RELATED APPLICATIONS

The present application claims priority to and the benefit of U.S. Provisional Application No. 62/636,391, filed Feb. 28, 2018, the content of which is incorporated by reference herein in its entirety.

### FIELD OF THE INVENTION

The invention generally relates to methods for assessing a risk of increased time-to-first-conception in a female. In particular, genetic variants are identified that are associated with a time to conception longer than 12 months for a first pregnancy among women less than 35 years of age.

### BACKGROUND

On average, couples of reproductive age in the United States achieve pregnancy within 6 months of starting timed intercourse. However, approximately 15% take longer than a year or are unable to conceive at all. According to the Centers for Disease Control and Prevention, 6.7 million women in the United States between the ages of 15 and 44 suffer from impaired fecundity. A woman's egg quality and number naturally begin to decline at around age 35. Reduced fecundity as a result of declining ovarian reserve and function leading up to menopause is a normal part of aging in females.

Maternal age is often cited as a major determinant of time to conception. However, in some women, ovarian aging happens prematurely, sometimes resulting in ovarian function disorders such as diminished ovarian reserve (DOR), primary ovarian insufficiency (POI), or polycystic ovary syndrome (PCOS), which have a negative impact on fecundity.

For women below the age of 35 who do not otherwise have signs of ovarian function disorders, the cause of increased time to conception often remains unknown. Other clinical or subclinical factors may contribute to the increased time to conception phenotype.

### SUMMARY

The present disclosure uncovers genetic factors that affect time to conception, which provide a better understanding of the causes of the phenotype and are therefore useful in driving better reproductive care and outcomes. The analysis shows that variants in genes that regulate uterine development, endometrial function, and gonadotropin signaling are associated with increased time to conception. In particular, two newly discovered loci are found to have a highly significant correlation with the phenotype. These loci are useful alone or in combination with other biomarkers and phenotypic markers to diagnose or assess the risk of an increased time to conception phenotype, and can help guide diagnosis and treatment while improving outcomes.

As explained in further detail below, the newly discovered associations form the basis for methods of diagnosing and treating infertility. The loci identified herein are useful as companion diagnostics, either alone or in combination with other known markers, for therapeutic response in patients observed to have an increased time to conception.

In certain aspects, the invention provides a method for assessing an increased time-to-conception phenotype in a female. The method involves obtaining a sample from a female subject and analyzing the sample to determine a presence of a biomarker, such as a gene or a gene product, associated with endometriosis, uterine development, endometrial function, or gonadotropin signaling. The method then entails determining a probability of increased time-to-conception based on the presence of the biomarker.

In some embodiments, analyzing the sample comprises sequencing a portion of the gene to determine a presence or absence of a variant. The variant may be a single nucleotide polymorphism, a deletion, an insertion, a rearrangement, a copy number variation, or a combination thereof. The biomarker may be associated with the WNT4 or FSHB genes. In embodiments where the biomarker is a gene product, the gene product may be RNA or protein. The sample may be human tissue or body fluid, including blood. The sample may include nucleic acid of the female subject.

In some embodiments, the method also includes the step of determining course of treatment based on the probability of increased time-to-conception. The treatment may include in vitro fertilization.

In other aspects, the invention provides a method for determining course of treatment to decrease time-to-conception. The method includes obtaining a sample from a female patient, determining the presence of a biomarker associated with delayed time-to-conception. The biomarker may be associated with endometriosis, uterine development, endometrial function, or gonadotropin signaling. The course of treatment is determined by the presence of a biomarker associated with delayed time-to-conception. The treatment may be in vitro fertilization (IVF), fertility drugs, or intrauterine insemination. In other aspects, the treatment may include vitro fertilization (IVF) or intrauterine insemination and fertility drugs. In other aspects, the dose of fertility drugs may be determined based on the presence of the biomarker.

In related aspects, the invention provides a method for identifying genetic factors associated with infertility in a female. The method includes obtaining sample genetic data from a plurality of females suspected of infertility, genotyping the sample genetic data on a genome-wide array, conducting a regression to correlate regions of the sample genetic data with an infertility trait such as a reported time to first conception of greater than twelve months, and identifying a locus among the regions that has a statistically significant correlation with the infertility trait. In certain embodiments, the genome-wide array includes biomarkers associated with endometriosis, uterine development, endometrial function, or gonadotropin signaling. The biomarkers may include WNT4 and FSHB.

In other aspects of the invention, an array is provided for assessing a time to conception phenotype. The array includes a substrate and a plurality of oligonucleotides attached to the substrate at discrete addressable positions, wherein at least one of the oligonucleotides hybridizes to a portion of a gene selected from the group including WNT4 and FSHB.

### BRIEF DESCRIPTION OF THE DRAWINGS

FIG. 1 illustrates a flowchart of the case-control ascertainment process for a genome-wide association study (GWAS) for time-to-conception longer than 12 months.

FIG. 2 depicts a quantile-quantile plot of association results from the GWAS.

FIG. 3 depicts a Manhattan plot of data showing two loci covering the WNT4 and FSHB genes reaching genome-wide significance.

FIG. 4 depicts a regional association plot of the 1p36.12 locus, which includes the WNT4 gene

FIG. 5 depicts a regional association plot of the 11p14.1 locus, which includes the FSHB gene.

FIG. 6 depicts a system for implementing methods of the invention.

FIG. 7 depicts the ESR1 binding site in WNT4 locus.

FIG. 8 depicts the LHX3 binding site in FSHB locus.

### DETAILED DESCRIPTION

The present disclosure relates to genetic variants associated with a risk of increased time-to-first-conception phenotype. Several variants were identified through a genome-wide association study that influence the time to first conception. Many of the variants are connected to mechanisms that regulate uterine development, endometrial function, and gonadotropin signaling. These newly discovered connections between certain loci and increased time to conception are useful individually or in combination with other known biomarkers and phenotypic markers to assess a prospective mother's fertility outlook. This information can therefore be used to guide treatment options such as in vitro fertilization or other therapies, including administration of certain drugs, for example, estrogen, The presence of certain biomarkers related to delayed time-to-conception may also be used to inform the course of treatment to improve or reduce time-to-conception and/or infertility.

Time-to-conception (TTC) is defined as the amount of time it takes a female to become pregnant after beginning to try to conceive. For example, a woman who begins trying to become pregnant in February and who conceives in May would have a TTC of approximately 3 months. Women who report conceiving within 6 months during their first attempt at pregnancy may be considered to have a normal TTC phenotype, whereas those who report trying to conceive for 13 months or more may be considered to have an increased TTC phenotype. Women whose TTC is between 6 and 13 months may be considered borderline. The precise TTC categories may be assigned different values, but in general the present disclosure recognizes that a longer TTC phenotype can indicate an underlying genotype. Accordingly, a genome-wide association study comparing two self-reported populations of women, one with an TTC of greater than 13 months and one a TTC of less than 6 months, was conducted to identify potential genetic bases for the increased TTC phenotype.

On this basis, variants were identified that were associated with increased time to conception. Patient samples were genotyped on one of four versions of a custom genome-wide genotyping array, targeting between 556,000 and 955,000 genetic variants. The samples were then imputed for 15 million variants using phase I of the 1000 Genomes Project as a reference. The data were analyzed using logistic regression under an additive model with age, top five principal components, and genotyping array version as covariates. Two genomic loci were genome-wide significant (p<5×10⁻⁸) with the phenotype of trying to conceive a first pregnancy taking longer than 13 months.

The first variant was in the WNT4 gene (variant rs61768001; p=4.6×10−10, OR=1.16). WNT4 has been linked to regulation of both uterine embryonic development and postnatal uterine biology. The variant rs3820282 has been shown to modulate the binding of Estrogen Receptor 1 (ESR1) in the WNT4 locus, with the allele associated with higher time-to-conception increasing the binding of ESR1. WNT4 is a key regulator of human endometrial decidualization, the process during which the endometrial stromal fibroblasts differentiate into specialized secretory decidual cells to enable embryo implantation and placental development. Reducing WNT4 expression in human endometrial stromal cells (HESCs) by small interfering RNA administration significantly hampers BMP2-induced stromal differentiation. Rare missense variants within WNT4 have been found in patients with Müllerian aplasia. Deletion of Wnt4 in the mouse uterus compromises embryo implantation and causes subfertility. The WNT4 locus has also been associated with endometriosis in published GWAS.

The second variant was ˜25 kb upstream of the FSHB gene (variant rs11031006; p=3.6×10−8, OR=1.14). FSHB encodes the β-subunit of follicle stimulating hormone (FSH). Another variant rs10835638, which is in the FSHB promoter, was a credible SNP in the analysis. This SNP has been associated with reduced FSH serum levels, PCOS diagnosis, longer menstrual cycles, increased age of menopause, and higher rates of female nulliparity. By contrast, the rs11031006-G allele, which was associated with conception in <6 months in our analysis, has been associated with earlier age at menarche, first live birth, and menopause, but also higher dizygotic twinning rates and higher lifetime parity.

The results of this analysis show that increased time to first conception is associated with variants in genes and pathways regulating uterine development, endometrial function, and gonadotropin signaling. The newly-discovered loci are also useful in combination with other biomarkers and phenotypic markers to diagnose or assess the risk of an increased TTC phenotype. As explained in further detail below, these associations form the basis for methods of diagnosing and treating infertility. The loci identified herein are useful as companion diagnostics, either alone or in combination with other known markers, for therapeutic response in patients observed to have an increased time to conception.

### Samples

Methods of the invention involve obtaining a sample (e.g., a tissue or body fluid) that is suspected to include a gene or gene product of interest. In particular embodiments, the sample is a maternal sample. The sample may be collected in any clinically acceptable manner. A tissue is a mass of connected cells and/or extracellular matrix material, e.g. skin tissue, hair, nails, endometrial tissue, nasal passage tissue, CNS tissue, neural tissue, eye tissue, liver tissue, kidney tissue, placental tissue, mammary gland tissue, placental tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, blood, maternal blood, phlegm, saliva, sweat, amniotic fluid, menstrual fluid, endometrial aspirates, mammary fluid, follicular fluid of the ovary, fallopian tube fluid, peritoneal fluid, urine, and cerebrospinal fluid (CSF), such as lumbar or ventricular CSF. A sample may also be a fine needle aspirate or biopsied tissue. A sample also may be media containing cells or biological material. A sample may also be a blood clot, for example, a blood clot that has been obtained from whole blood after the serum has been removed. In certain embodiments, genes or gene products associated with an increased TTC phenotype may be found in reproductive cells or tissues, such as gametic cells, gonadal tissue, fertilized embryos, and placenta. In certain embodiments, the sample is drawn maternal blood or saliva.

Nucleic acid is extracted from the sample according to methods known in the art. See for example, Maniatis, et al., Molecular Cloning: A Laboratory Manual, Cold Spring Harbor, N.Y., pp. 280-281, 1982, the contents of which are incorporated by reference herein in their entirety. In certain embodiments, a genomic sample is collected from a subject followed by enrichment for genetic regions or genetic fragments of interest, for example by hybridization to a nucleotide array comprising fertility-related genes or gene fragments of interest. The sample may be enriched for genes of interest (e.g., infertility-associated genes) using methods known in the art, such as hybrid capture. See for examples, Lapidus (U.S. Pat. No. 7,666,593), the content of which is incorporated by reference herein in its entirety.

RNA may be isolated from eukaryotic cells by procedures that involve lysis of the cells and denaturation of the proteins contained therein. Tissue of interest includes gametic cells, gonadal tissue, endometrial tissue, fertilized embryos, and placenta. RNA may be isolated from fluids of interest by procedures that involve denaturation of the proteins contained therein. Fluids of interest include blood, menstrual fluid, mammary fluid, follicular fluid of the ovary, peritoneal fluid, or culture medium. Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsC1 centrifugation to separate the RNA from DNA (Chirgwin et al., Biochemistry 18:5294-5299 (1979)). Poly(A)+RNA is selected by selection with oligo-dT cellulose (see Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol. If desired, RNase inhibitors may be added to the lysis buffer. Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or SEPHADEX (see Ausubel et al., CURRENT PROTOCOLS IN MOLECULAR BIOLOGY, vol. 2, Current Protocols Publishing, New York (1994). Once bound, poly(A)+mRNA is eluted from the affinity column using 2 mM EDTA/0.1% SDS.

Genetic data can be obtained, for example, by conducting an assay that detects a variant in an infertility-associated genetic region or abnormal expression of an infertility-associated genetic region. The presence of certain variants in those genetic regions or abnormal expression levels of those genetic regions is indicative of fertility- or fecundity-related disorders. Exemplary variants include, but are not limited to, a single nucleotide polymorphism, a single nucleotide variant, a deletion, an insertion, an inversion, a genetic rearrangement, a copy number variation, chromosomal microdeletion, genetic mosaicism, karyotype abnormality, or a combination thereof.

In particular embodiments, the assay is conducted on genetic regions of fertility related genes, or more specifically, genetic regions related to endometriosis, uterine development, endometrial function, or gonadotropin signaling. Detailed descriptions of conventional methods, such as those employed to make and use nucleic acid arrays, amplification primers, hybridization probes, and the like are found in standard laboratory manuals such as: Genome Analysis: A Laboratory Manual Series (Vols. I-IV), Cold Spring Harbor Laboratory Press; PCR Primer: A Laboratory Manual, Cold Spring Harbor Laboratory Press; and Sambrook, J et al., (2001) Molecular Cloning: A Laboratory Manual, 2nd ed. (Vols. 1-3), Cold Spring Harbor Laboratory Press. Custom nucleic acid arrays are commercially available from, e.g., Affymetrix (Santa Clara, Calif.), Applied Biosystems (Foster City, Calif.), and Agilent Technologies (Santa Clara, Calif.).

Methods of detecting genomic variants are known in the art. In certain embodiments, a known single nucleotide polymorphism at a particular position can be detected by single base extension for a primer that binds to the sample DNA adjacent to that position. See for example Shuber et al. (U.S. Pat. No. 6,566,101), the content of which is incorporated by reference herein in its entirety. In other embodiments, a hybridization probe might be employed that overlaps the SNP of interest and selectively hybridizes to sample nucleic acids containing a particular nucleotide at that position. See for example Shuber et al. (U.S. Pat. Nos. 6,214,558 and 6,300,077), the content of which is incorporated by reference herein in its entirety.

In particular embodiments, nucleic acids are sequenced in order to detect variants (i.e., mutations) in the nucleic acids compared to wild-type and/or non-mutated forms of the sequence. Methods of detecting sequence variants are known in the art and are described herein. Sequence reads can be analyzed to call variants by any number of methods known in the art. Variant calling can include aligning sequence reads to a reference (e.g. hg18) and reporting single nucleotide (SNP) alleles. An example of methods for analyzing sequence reads and calling variants includes standard Genome Analysis Toolkit (GATK) methods. See The Genome Analysis Toolkit: a MapReduce framework for analyzing next-generation DNA sequencing data, Genome Res 20(9):1297-1303, the contents of each of which are incorporated by reference. GATK is a software package for analysis of high-throughput sequencing data capable of identifying variants, including SNPs.

### Biomarkers

A biomarker generally refers to a molecule that may act as an indicator of a biological state. Biomarkers for use with methods of the invention may be any marker that is associated with infertility. Exemplary biomarkers include genes (e.g. any region of DNA encoding a functional product), genetic regions (e.g. regions including genes and intergenic regions with a particular focus on regions conserved throughout evolution in placental mammals), and gene products (e.g., RNA and protein). In certain embodiments, the biomarker is an infertility-associated genetic region. An infertility-associated genetic region is any DNA sequence in which variation is associated with a change in fertility. Examples of changes in fertility include, but are not limited to, the following: a homozygous mutation of an infertility-associated gene leads to a complete loss of fertility; a homozygous variant of an infertility-associated gene is incompletely penetrant and leads to reduction in fertility that varies from individual to individual; a heterozygous variant is completely recessive, having no effect on fertility; and the infertility-associated gene is X-linked, such that a potential defect in fertility depends on whether a non-functional allele of the gene is located on an inactive X chromosome (Barr body) or on an expressed X chromosome.

In particular embodiments, the infertility-associated genetic region is a maternal effect gene. Maternal effects genes are genes that have been found to encode key structures and functions in mammalian oocytes (Yurttas et al., Reproduction 139:809-823, 2010). Maternal effect genes are described, for example in, Christians et al. (Mol Cell Biol 17:778-88, 1997); Christians et al., Nature 407:693-694, 2000); Xiao et al. (EMBO J 18:5943-5952, 1999); Tong et al. (Endocrinology 145:1427-1434, 2004); Tong et al. (Nat Genet 26:267-268, 2000); Tong et al. (Endocrinology, 140:3720-3726, 1999); Tong et al. (Hum Reprod 17:903-911, 2002); Ohsugi et al. (Development 135:259-269, 2008); Borowczyk et al. (Proc Natl Acad Sci U S A., 2009); and Wu (Hum Reprod 24:415-424, 2009). The content of each of these is incorporated by reference herein in its entirety.

In particular embodiments, the infertility-associated genetic region is a gene (including regions flanking a gene within about 25 kb on either side of the gene) that regulates uterine development, endometrial function, or gonadotropin signaling. The genetic region may be in or near the WNT4 or FSHB genes.

In particular embodiments, the infertility-associated genetic region is a gene (including exons, introns, and 10 kb of DNA flanking either side of said gene) selected from the genes shown in Table 1 below. In Table 1, OMIM reference numbers are provided when available.

TABLE 1
Human Infertility-Related Genes (OMIM #)

| | | | |
|---|---|---|---|
| ABCA1 (600046) | ACTL6A (604958) | ACTL8 | ACVR1 (102576) |
| ACVR1B (601300) | ACVR1C (608981) | ACVR2(102581) | ACVR2A (102581) |
| ACVR2B (602730) | ACVRL1 (601284) | ADA (608958) | ADAMTS1 (605174) |
| ADM (103275) | ADM2 (608682) | AFF2 (300806) | AGT (106150) |
| AHR (600253) | AIRE (607358) | AK2 (103020) | AK7 |
| AKR1C1 (600449) | AKR1C2 (600450) | AKR1C3 (603966) | AKR1C4 (600451) |
| AKT1 (164730) | ALDOA (103850) | ALDOB (612724) | ALDOC (103870) |
| ALPL (171760) | AMBP (176870) | AMD1 (180980) | AMH (600957) |
| AMHR2 (600956) | ANK3 (600465) | ANXA1 (151690) | APC (611731) |
| APOA1 (107680) | APOE (107741) | AQP4 (600308) | AR (313700) |
| AREG (104640) | ARF1 (103180) | ARF3 (103190) | ARF4 (601177) |
| ARF5 (103188) | ARFRP1 (604699) | ARL1 (603425) | ARL10 (612405) |
| ARL11 (609351) | ARL13A | ARL13B (608922) | ARL15 |
| ARL2 (601175) | ARL3 (604695) | ARL4A (604786) | ARL4C (604787) |
| ARL4D (600732) | ARL5A (608960) | ARL5B (608909) | ARL5C |
| ARL6 (608845) | ARL8A | ARL8B | ARMC2 |
| ARNTL (602550) | ASCL2 (601886) | ATF71P (613644) | ATG7 (608760) |
| ATM (607585) | ATR (601215) | ATXN2 (601517) | AURKA (603072) |
| AURKB (604970) | AUTS2 (607270) | BARD1 (601593) | BAX (600040) |
| BBS1 (209901) | BBS10 (610148) | BBS12 (610683) | BBS2 (606151) |
| BBS4 (600374) | BBS5 (603650) | BBS7 (607590) | BBS9 (607968) |
| BCL2 (151430) | BCL2L1 (600039) | BCL2L10 (606910) | BDNF (113505) |
| BECN1 (604378) | BHMT (602888) | BLVRB (600941) | BMP15 (300247) |
| BMP2 (112261) | BMP3 (112263) | BMP4 (112262) | BMP5 (112265) |
| BMP6 (112266) | BMP7 (112267) | BMPR1A (601299) | BMPR1B (603248) |
| BMPR2 (600799) | BNC1 (601930) | BOP1 (610596) | BRCA1 (113705) |
| BRCA2 (600185) | BRIP1 (605882) | BRSK1 (609235) | BRWD1 |
| BSG (109480) | BTG4 (605673) | BUB1 (602452) | BUB1B (602860) |
| C2orf86 (613580) | C3 (120700) | C3orf56 | C6orf221 (611687) |
| CA1 (114800) | CARDS (609051) | CARM1 (603934) | CASP1 (147678) |
| CASP2 (600639) | CASP5 (602665) | CASP6 (601532) | CASP8 (601763) |
| CBS (613381) | CBX1 (604511) | CBX2 (602770) | CBX5 (604478) |
| CCDC101 (613374) | CCDC28B (610162) | CCL13 (601391) | CCL14 (601392) |
| CCL4 (182284) | CCL5 (187011) | CCL8 (602283) | CCND1 (168461) |
| CCND2 (123833) | CCND3 (123834) | CCNH (601953) | CCS (603864) |
| CD19 (107265) | CD24 (60007 4) | CD55 (125240) | CD81 (186845) |
| CD9 (143030) | CDC42 (116952) | CDK4 (123829) | CDK6 (603368) |
| CDK7 (601955) | CDKN1B (600778) | CDKN1C (600856) | CDKN2A (600160) |
| CDX2 (600297) | CDX4 (300025) | CEACAM20 | CEBPA (116897) |
| CEBPB (189965) | CEBPD (116898) | CEBPE (600749) | CEBPG (138972) |
| CEBPZ (612828) | CELF1 (601074) | CELF4 (612679) | CENPB (117140) |
| CENPF (600236) | CENPI (300065) | CEP290 (610142) | CFC1 (605194) |
| CGA (118850) | CGB (118860) | CGB1 (608823) | CGB2 (608824) |
| CGB5 (608825) | CHD7 (608892) | CHST2 (603798) | CLDN3 (602910) |
| COIL (600272) | COL1A2 (120160) | COL4A3BP (604677) | COMT (116790) |
| COPE (606942) | COX2 (600262) | CP (117700) | CPEB1 (607342) |
| CRHR1 (122561) | CRYBB2 (123620) | CSF1 (120420) | CSF2 (138960) |
| CSTF1 (600369) | CSTF2 (600368) | CTCF (604167) | CTCFL (607022) |
| CTF2P | CTGF (121009) | CTH (607657) | CTNNB1 (116806) |
| CUL1 (603134) | CX3CL1 (601880) | CXCL10 (147310) | CXCL9 (601704) |
| CXorf67 | CYP11A1 (118485) | CYP11B1 (610613) | CYP11B2 (124080) |
| CYP17A1 (609300) | CYP19A1 (107910) | CYP1A1 (108330) | CYP27B1 (609506) |
| DAZ2 (400026) | DAZL (601486) | DCTPP1 | DDIT3 (126337) |
| DDX11 (601150) | DDX20 (606168) | DDX3X (300160) | DDX43 (606286) |
| DEPDC7 (612294) | DHFR (126060) | DHFRL1 | DIAPH2 (300108) |
| DICER1 (606241) | DKK1 (605189) | DLC1 (604258) | DLGAP5 |
| DMAP1 (605077) | DMC1 (602721) | DNAJB1 (604572) | DNMT1 (126375) |
| DNMT3B (602900) | DPPA3 (608408) | DPPA5 (611111) | DPYD (612779) |
| DTNBP1 (607145) | DYNLL1 (601562) | ECHS1 (602292) | EEF1A1 (130590) |
| EEF1A2 (602959) | EFNA1 (191164) | EFNA2 (602756) | EFNA3 (601381) |
| EFNA4 (601380) | EFNA5 (601535) | EFNB1 (300035) | EFNB2 (600527) |
| EFNB3 (602297) | EGR1 (128990) | EGR2 (129010) | EGR3 (602419) |
| EGR4 (128992) | EHMT1 (607001) | EHMT2 (604599) | EIF2B2 (606454) |
| EIF2B4 (606687) | EIF2B5 (603945) | EIF2C2 (606229) | EIF3C (603916) |
| EIF3CL (603916) | EPHA1 (179610) | EPHA10 (611123) | EPHA2 (176946) |
| EPHA3 (179611) | EPHA4 (602188) | EPHA5 (600004) | EPHA6 (600066) |
| EPHA7 (602190) | EPHA8 (176945) | EPHB1 (600600) | EPHB2 (600997) |
| EPHB3 (601839) | EPHB4 (6000 11) | EPHB6 (602757) | ERCC1 (126380) |
| ERCC2 (126340) | EREG (602061) | ESR1 (133430) | ESR2 (601663) |
| ESR2 (601663) | ESRRB (602167) | ETV5 (601600) | EZH2 (601573) |
| EZR (123900) | FANCC (613899) | FANCG (602956) | FANCL (608111) |
| FAR1 | FAR2 | FASLG (134638) | FBN1 (134797) |
| FBN2 (612570) | FBN3 (608529) | FBRS (608601) | FBRSL1 |
| FBXO10 (609092) | FBXO11 (607871) | FCRL3 (606510) | FDXR (103270) |
| FGF23 (605380) | FGF8 (600483) | FGFBP1 (607737) | FGFBP3 |
| FGFR1 (136350) | FHL2 (602633) | FIGLA (608697) | FILIP1L (612993) |
| FKBP4 (600611) | FMN2 (606373) | FMR1 (309550) | FOLR1 (136430) |
| FOLR2 (136425) | FOXE1 (602617) | FOXL2 (605597) | FOXN1 (600838) |
| FOXO3 (602681) | FOXP3 (300292) | FRZB (605083) | FSHB (136530) |
| FSHR (136435) | FST (136470) | GALT (606999) | GBP5 (611467) |
| GCK (138079) | GDF1 (602880) | GDF3 (606522) | GDF9 (601918) |
| GGT1 (612346) | GJA1 (121014) | GJA10 (611924) | GJA3 (121015) |
| GJA4 (121012) | GJA5 (121013) | GJA8 (600897) | GJB1 (304040) |
| GJB2 (121011) | GJB3 (603324) | GJB4 (605425) | GJB6 (604418) |
| GJB7 (611921) | GJC1 (608655) | GJC2 (608803) | GJC3 (611925) |
| GJD2 (607058) | GJD3 (607425) | GJD4 (611922) | GNA13 (604406) |
| GNB2 (139390) | GNRH1 (152760) | GNRH2 (602352) | GNRHR (138850) |
| GPC3 (300037) | GPRC5A (604138) | GPRC5B (605948) | GREM2 (608832) |
| GRN (138945) | GSPT1 (139259) | GSTA1 (138359) | H19 (103280) |
| H1FOO (142709) | HABP2 (603924) | HADHA (600890) | HAND2 (602407) |
| HBA1 (141800) | HBA2 (141850) | HBB (141900) | HELLS (603946) |
| HK3 (142570) | HMOX1 (141250) | HNRNPK (600712) | HOXA11 (142958) |
| HPGD (601688) | HS6ST1 (604846) | HSD17B1 (109684) | HSD17B12 (609574) |
| HSD17B2 (1 09685) | HSD17B4 (601860) | HSD17B7 (606756) | HSD3B1 (109715) |
| HSP1 (140580) | HSP2BP (604554) | HSP90B1 (191175) | HSPG2 (142461) |
| HTATIP2 (605628) | ICAM1 (147840) | ICAM2 (146630) | ICAM3 (146631) |
| IDH1 (147700) | IFI30 (604664) | IFITM1 (604456) | IGF1 (147440) |
| IGP1R (147370) | IGP2 (147470) | IGF2BP1 (608288) | IGF2BP2 (608289) |
| IGP2BP3 (608259) | IGP2BP3 (608259) | IGF2R (147280) | IGFALS (601489) |
| IGPBP1 (146730) | IGPBP2 (146731) | IGFBP3 (146732) | IGFBP4 (146733) |
| IGPBP5 (146734) | IGPBP6 (146735) | IGFBP7 (602867) | IGFBPL1 (610413) |
| IL10 (124092) | IL11RA (600939) | IL12A (161560) | IL12B (161561) |
| IL13 (147683) | IL17A (603149) | IL17B (604627) | IL17C (604628) |
| IL17D (607587) | IL17P (606496) | IL1A (147760) | IL1B (147720) |
| IL23A (605580) | IL23R (607562) | IL4 (147780) | IL5 (147850) |
| IL5RA (147851) | IL6 (147620) | IL6ST (600694) | IL8 (146930) |
| ILK (602366) | INHA (147380) | INHBA (147290) | INHBB (147390) |
| IRP1 (147575) | ISG15 (147571) | ITGA11 (604789) | ITGA2 (192974) |
| ITGA3 (605025) | ITGA4 (192975) | ITGA7 (600536) | ITGA9 (603963) |
| ITGAV (193210) | ITGB1 (135630) | JAG1 (601920) | JAG2 (602570) |
| JARID2 (601594) | JMY (604279) | KAL1 (300836) | KDM1A (609132) |
| KDM1B (613081) | KDM3A (611512) | KDM4A (609764) | KDM5A (180202) |
| KDM5B (605393) | KHDC1 (611688) | KIAA0430 (614593) | KIF2C (604538) |
| KISS1 (603286) | KISS1R (604161) | KITLG (184745) | KL (604824) |
| KLF4 (602253) | KLF9 (602902) | KLHL7 (611119) | LAMC1 (150290) |
| LAMC2 (150292) | LAMP1 (153330) | LAMP2 (309060) | LAMP3 (605883) |
| LDB3 (605906) | LEP (164160) | LEPR (601007) | LPNG (602576) |
| LHB (152780) | LHCGR (152790) | LHX8 (604425) | LIF (159540) |
| LIFR (151443) | LIMS1 (602567) | LIMS2 (607908) | LIMS3 |
| LIMS3L | LIN28 (611043) | LIN28B (611044) | LMNA (150330) |
| LOC613037 | LOXL4 (607318) | LPP (600700) | LYRM1 (614709) |
| MADIL1 (602686) | MAD2L1 (601467) | MAD2L1BP | MAF (177075) |
| MAP3K1 (600982) | MAP3K2 (609487) | MAPK1 (176948) | MAPK3 (601795) |
| MAPK8 (601158) | MAPK9 (602896) | MB21D1 (613973) | MBD1 (156535) |
| MBD2 (603547) | MBD3 (603573) | MBD4 (603574) | MCL1 (159552) |
| MCM8 (608187) | MDK (162096) | MDM2 (164785) | MDM4 (602704) |
| MECP2 (300005) | MED12 (300188) | MERTK (604705) | METTL3 (612472) |
| MGAT1 (160995) | MITF (156845) | MKKS (604896) | MKS1 (609883) |
| MLH1 (120436) | MLH3 (604395) | MOS (190060) | MPPED2 (600911) |
| MRS2 | MSH2 (609309) | MSH3 (600887) | MSH4 (602105) |
| MSH5 (603382) | MSH6 (600678) | MST1 (142408) | MSX1 (142983) |
| MSX2 (123101) | MTA2 (603947) | MTHFD1 (172460) | MTHFR (607093) |
| MTO1 (614667) | MTOR (601231) | MTRR (602568) | MUC4 (158372) |
| MVP (605088) | MX1 (147150) | MYC (190080) | NAB1 (600800) |
| NAB2 (602381) | NAT1 (108345) | NCAM1 (116930) | NCOA2 (601993) |
| NCOR1 (600849) | NCOR2 (600848) | NDP (300658) | NFE2L3 (604135) |
| NLRP1 (606636) | NLRP10 (609662) | NLRP11 (609664) | NLRP12 (609648) |
| NLRP13 (609660) | NLRP14 (609665) | NLRP2 (609364) | NLRP3 (606416) |
| NLRP4 (609645) | NLRP5 (609658) | NLRP6 (609650) | NLRP7 (609661) |
| NLRP8 (609659) | NLRP9 (609663) | NNMT (600008) | NOBOX (610934) |
| NODAL (601265) | NOG (602991) | NOS3 (163729) | NOTCH1 (190198) |
| NOTCH2 (600275) | NPM2 (608073) | NPR2 (108961) | NR2C2 (601426) |
| NR3C1 (138040) | NR5A1 (184757) | NR5A2 (604453) | NRIP1 (602490) |
| NRIP2 | NRIP3 (613125) | NTF4 (162662) | NTRK1 (191315) |
| NTRK2 (600456) | NUPR1 (614812) | OAS1 (164350) | OAT (613349) |
| OFD1 (300170) | OOEP (611689) | ORAI1 (610277) | OTC (300461) |
| PADI1 (607934) | PADI2 (607935) | PADI3 (606755) | PADI4 (605347) |
| PADI6 (610363) | PAEP (173310) | PAIP1 (605184) | PARP12 (612481) |
| PCNA (176740) | PCP4L1 | PDE3A (123805) | PDK1 (602524) |
| PGK1 (311800) | PGR (607311) | PGRMC1 (300435) | PGRMC2 (607735) |
| PIGA (311770) | PIM1 (164960) | PLA2G2A (172411) | PLA2G4C (603602) |
| PLA2G7 (601690) | PLAC1L | PLAG1 (603026) | PLAGL1 (603044) |
| PLCB1 (607120) | PMS1 (600258) | PMS2 (600259) | POF1B (300603) |
| POLG (174763) | POLR3A (614258) | POMZP3 (600587) | POU5F1 (164177) |
| PPID (601753) | PPP2CB (176916) | PRDM1 (603423) | PRDM9 (609760) |
| PRKCA (176960) | PRKCB (176970) | PRKCD (176977) | PRKCDBP |
| PRKCE (176975) | PRKCG (176980) | PRKCQ (600448) | PRKRA (603424) |
| PRLR (176761) | PRMT1 (602950) | PRMT10 (307150) | PRMT2 (601961) |
| PRMT3 (603190) | PRMT5 (604045) | PRMT6 (608274) | PRMT7 (610087) |
| PRMT8 (610086) | PROK1 (606233) | PROK2 (607002) | PROKR1 (607122) |
| PROKR2 (607123) | PSEN1 (104311) | PSEN2 (600759) | PTGDR (604687) |
| PTGER1 (176802) | PTGER2 (176804) | PTGER3 (176806) | PTGER4 (601586) |
| PTGES (605172) | PTGES2 (608152) | PTGES3 (607061) | PTGFR (600563) |
| PTGFRN (601204) | PTGS1 (176805) | PTGS2 (600262) | PTN (162095) |
| PTX3 (602492) | QDPR (612676) | RAD17 (603139) | RAX (601881) |
| RBP4 (180250) | RCOR1 (607675) | RCOR2 | RCOR3 |
| RDH11 (607849) | REC8 (608193) | REXO1 (609614) | REXO2 (607149) |
| RFPL4A (612601) | RGS2 (600861) | RGS3 (602189) | RSPO1 (609595) |
| RTEL1 (608833) | SAFB (602895) | SAR1A (607691) | SAR1B (607690) |
| SCARB1 (601040) | SDC3 (186357) | SELL (153240) | SEPHS1 (600902) |
| SEPHS2 (606218) | SERPINA10 (605271) | SFRP1 (604156) | SFRP2 (604157) |
| SFRP4 (606570) | SFRP5 (604158) | SGK1 (602958) | SGOL2 (612425) |
| SH2B1 (608937) | SH2B2 (605300) | SH2B3 (605093) | SIRT1 (604479) |
| SIRT2 (604480) | SIRT3 (604481) | SIRT4 (604482) | SIRT5 (604483) |
| SIRT6 (606211) | SIRT7 (606212) | SLC19A1 (600424) | SLC28A1 (606207) |
| SLC28A2 (606208) | SLC28A3 (608269) | SLC2A8 (605245) | SLC6A2 (163970) |
| SLC6A4 (182138) | SLCO2A1 (601460) | SLITRK4 (300562) | SMAD1 (601595) |
| SMAD2 (601366) | SMAD3 (603109) | SMAD4 (600993) | SMAD5 (603110) |
| SMAD6 (602931) | SMAD7 (602932) | SMAD9 (603295) | SMARCA4 (603254) |
| SMARCA5 (603375) | SMC1A (300040) | SMC1B (608685) | SMC3 (606062) |
| SMC4 (605575) | SMPD1 (607608) | SOCS1 (603597) | SOD1 (147450) |
| SOD2 (147460) | SOD3 (185490) | SOX17 (610928) | SOX3 (313430) |
| SPAG17 | SPARC (182120) | SPIN1 (609936) | SPN (182160) |
| SPO11 (605114) | SPP1 (166490) | SPSB2 (611658) | SPTB (182870) |
| SPTBN1 (182790) | SPTBN4 (606214) | SRCAP (611421) | SRD5A1 (184753) |
| SRSF4 (601940) | SRSF7 (600572) | ST5 (140750) | STAG3 (608489) |
| STAR (600617) | STARD10 | STARD13 (609866) | STARD3 (607048) |
| STARD3NL (611759) | STARD4 (607049) | STARD5 (607050) | STARD6 (607051) |
| STARD7 | STARD8 (300689) | STARD9 (614642) | STAT1 (600555) |
| STAT2 (600556) | STAT3 (102582) | STAT4 (600558) | STAT5A (601511) |
| STAT5B (604260) | STAT6 (601512) | STC1 (601185) | STIM1 (605921) |
| STK3 (605030) | SULT1E1 (600043) | SUZ12 (606245) | SYCE1 (611486) |
| SYCE2 (611487) | SYCP1 (602162) | SYCP2 (604105) | SYCP3 (604759) |
| SYNE1 (608441) | SYNE2 (608442) | TAC3 (162330) | TACC3 (605303) |
| TACR3 (162332) | TAF10 (600475) | TAF3 (606576) | TAF4 (601796) |
| TAF4B (601689) | TAF5 (601787) | TAF5L | TAF8 (609514) |
| TAF9 (600822) | TAP1 (170260) | TBL1X (300196) | TBXA2R (188070) |
| TCL1A (186960) | TCL1B (603769) | TCL6 (604412) | TCN2 (613441) |
| TDGF1 (187395) | TERC (602322) | TERF1 (600951) | TERT (187270) |
| TEX12 (605791) | TEX9 | TF (190000) | TFAP2C (601602) |
| TFPI (152310) | TFPI2 (600033) | TG (188450) | TGFB1 (190180) |
| TGFB1I1 (602353) | TGFBR3 (600742) | THOC5 (612733) | THSD7B |
| TLE6 (612399) | TM4SF1 (191155) | TMEM67 (609884) | TNF (191160) |
| TNFAIP6 (600410) | TNFSF13B (603969) | TOP2A (126430) | TOP2B (126431) |
| TP53 (191170) | TP53I3 (605171) | TP63 (603273) | TP73 (601990) |
| TPMT (187680) | TPRXL (611167) | TPT1 (600763) | TRIM32 (602290) |
| TSC2 (191092) | TSHB (188540) | TSIX (300181) | TTC8 (608132) |
| TUBB4Q (158900) | TUFM (602389) | TYMS (188350) | UBB (191339) |
| UBC (191340) | UBD (606050) | UBE2D3 (602963) | UBE3A (601623) |
| UBL4A (312070) | UBL4B (611127) | UIMC1 (609433) | UQCR11 (609711) |
| UQCRC2 (191329) | USP9X (300072) | VDR (601769) | VEGFA (192240) |
| VEGFB (601398) | VEGFC (601528) | VHL (608537) | VIM (193060) |
| VKORC1 (608547) | VKORC1L1 (608838) | WAS (300392) | WISP2 (603399) |
| WNT7A (601570) | WNT7B (601967) | WT1 (607102) | XDH (607633) |
| XIST (314670) | YBX1 (154030) | YBX2 (611447) | ZAR1 (607520) |
| ZFX (314980) | ZNF22 (194529) | ZNF267 (604752) | ZNF689 |
| ZNF720 | ZNF787 | ZNF84 | ZP1 (195000) |
| ZP2 (182888) | ZP3 (182889) | ZP4 (613514) | |

The molecular products of the genes in Table 1 are involved in different aspects of oocyte and embryo physiology from transcription and chromosome remodeling to RNA processing and binding. Some highly conserved maternal effect genes are described below.

Peptidylarginine deiminase 6 (PADI6) Padi6 was originally cloned from a 2D murine egg proteome gel based on its relative abundance, and Padi6 expression in mice appears to be almost entirely limited to the oocyte and pre-implantation embryo (Yurttas et al., 2010). Padi6 is first expressed in primordial oocyte follicles and persists, at the protein level, throughout pre-implantation development to the blastocyst stage (Wright et al., Dev Biol, 256:73-88, 2003). Inactivation of Padi6 leads to female infertility in mice, with the Padi6-null developmental arrest occurring at the two-cell stage (Yurttas et al., 2008).

Nucleoplasmin 2 (NPM2) Nucleoplasmin is another maternal effect gene, and is thought to be phosphorylated during mouse oocyte maturation. NPM2 exhibits a phosphate sensitive increase in mass during oocyte maturation. Increased phosphorylation is retained through the pronuclear stage of development. NPM2 then becomes dephosphorylated at the two- cell stage and remains in this form throughout the rest of pre-implantation development. Further, its expression pattern appears to be restricted to oocytes and early embryos. Immunofluorescence analysis of NPM2 localization shows that NPM2 primarily localizes to the nucleus in mouse oocytes and early embryos. In mice, maternally-derived NPM2 is required for female fertility (Burns et al., 2003).

Maternal antigen the embryos require (MATER/NLRP5) MATER, the protein encoded by the Nlrp5 gene, is another highly abundant oocyte protein that is essential in mouse for embryonic development beyond the two-cell stage. MATER was originally identified as an oocyte-specific antigen in a mouse model of autoimmune premature ovarian failure (Tong et al., Endocrinology, 140:3720-3726, 1999). MATER demonstrates a similar expression and subcellular expression profile to PADI6. Like Padi6-null animals, Nlrp5-null females exhibit normal oogenesis, ovarian development, oocyte maturation, ovulation and fertilization. However, embryos derived from Nlrp5-null females undergo a developmental block at the two-cell stage and fail to exhibit normal embryonic genome activation (Tong et al., Nat Genet 26:267-268, 2000; and Tong et al. Mamm Genome 11:281-287, 2000b).

Brahma-related gene 1 (BRGI) Mammalian SWI/SNF-related chromatin remodeling complexes regulate transcription and are believed to be involved in zygotic genome activation (ZGA). Such complexes are composed of approximately nine subunits, which can be variable depending on cell type and tissue. The BRG1 catalytic subunit exhibits DNA-dependent APTase activity, and the energy derived from ATP hydrolysis alters the conformation and position of nucleosomes. Brg 1 is expressed in oocytes and has been shown to be essential in the mouse as null homozygotes do not progress beyond the blastocyst stage (Bultman et al., 2000).

Factor located in oocytes permitting embryonic development (FLOPED/OOEP) The subcortical maternal complex (SCMC) is a poorly characterized murine oocyte structure to which several maternal effect gene products localize (Li et al. Dev Cell 15:416-425, 2008). PADI6, MATER, FILIA, TLE6, and FLOPED have been shown to localize to this complex (Li et al. Dev Cell 15:416-425, 2008; Yurttas et al. Development 135:2627-2636, 2008). This complex is not present in the absence of Floped and Nlrp5, and similar to embryos resulting from Nlrp5-depleted oocytes, embryos resulting from Floped-null oocytes do not progress past the two cell stage of mouse development (Li et al., 2008). FLOPED is a small (19kD) RNA binding protein that has also been characterized under the name of MOEP19 (Herr et al., Dev Biol 314:300-316, 2008).

KH domain containing 3-like, subcortical maternal complex member (FILIA/KHDC3L) FILIA is another small RNA-binding domain containing maternally inherited murine protein. FILIA was identified and named for its interaction with MATER (Ohsugi et al. Development 135:259-269, 2008). Like other components of the SCMC, maternal inheritance of the Khdc3 gene product is required for early embryonic development. In mice, loss of Khdc3 results in a developmental arrest of varying severity with a high incidence of aneuploidy due, in part, to improper chromosome alignment during early cleavage divisions (Li et al., 2008). Khdc3 depletion also results in aneuploidy, due to spindle checkpoint assembly (SAC) inactivation, abnormal spindle assembly, and chromosome misalignment (Zheng et al. Proc Natl Acad Sci USA 106:7473-7478, 2009).

Basonuclin (BNCI) Basonuclin is a zinc finger transcription factor that has been studied in mice. It is found expressed in keratinocytes and germ cells (male and female) and regulates rRNA (via polymerase I) and mRNA (via polymerase II) synthesis (luchi and Green, 1999; Wang et al., 2006). Depending on the amount by which expression is reduced in oocytes, embryos may not develop beyond the 8-cell stage. In Bsnl depleted mice, a normal number of oocytes are ovulated even though oocyte development is perturbed, but many of these oocytes cannot go on to yield viable offspring (Ma et al., 2006).

Zygote Arrest 1 (ZAR1) Zar1 is an oocyte-specific maternal effect gene that is known to function at the oocyte to embryo transition in mice. High levels of Zar1 expression are observed in the cytoplasm of murine oocytes, and homozygous-null females are infertile: growing oocytes from Zar 1-null females do not progress past the two-cell stage.

Cytosolic phospholipase A2γ (PLA2G4C) Under normal conditions, cPLA2γ, the protein product of the murine PLA2G4C ortholog, expression is restricted to oocytes and early embryos in mice. At the subcellular level, cPLA2γ mainly localizes to the cortical regions, nucleoplasm, and multivesicular aggregates of oocytes. It is also worth noting that while cPLA2γ expression does appear to be mainly limited to oocytes and pre-implantation embryos in healthy mice, expression is considerably up-regulated within the intestinal epithelium of mice infected with Trichinella spiralis. This suggests that cPLA2γ may also play a role in the inflammatory response. The human PLA2G4C differs in that rather than being abundantly expressed in the ovary, it is abundantly expressed in the heart and skeletal muscle. Also, the human protein contains a lipase consensus sequence but lacks a calcium-binding domain found in other PLA2 enzymes. Accordingly, another cytosolic phospholipase may be more relevant for human fertility.

Transforming, Acidic Coiled-Coil Containing Protein 3 (TACC3) In mice, TACC3 is abundantly expressed in the cytoplasm of growing oocytes, and is required for microtubule anchoring at the centrosome and for spindle assembly and cell survival (Fu et al., 2010).

In certain embodiments, the gene is a gene that is expressed in an oocyte. Exemplary genes include CTCF, ZFP57, POU5F1, SEBOX, and HDAC1.

In other embodiments, the gene is a gene that is involved in DNA repair pathways, including but not limited to, MLH1, PMS1 and PMS2. In other embodiments, the gene is BRCA1 or BRCA2.

In other embodiments, the biomarker is a gene product (e.g., RNA or protein) of an infertility-associated gene. In particular embodiments, the gene product is a gene product of a maternal effect gene. In other embodiments, the gene product is a product of a gene from Table 1. In certain embodiments, the gene product is a product of a gene that is expressed in an oocyte, such as a product of CTCF, ZFP57, POU5F1, SEBOX, and HDAC1. In other embodiments, the gene product is a product of a gene that is involved in DNA repair pathways, such as a product of MLH1, PMS1, or PMS2. In other embodiments, gene product is a product of BRCA1 or BRCA2.

In other embodiments, the biomarker may be an epigenetic factor, such as methylation patterns (e.g., hypermethylation of CpG islands), genomic localization or post-translational modification of histone proteins, or general post-translational modification of proteins such as acetylation, ubiquitination, phosphorylation, or others.

In other embodiments, methods of the invention analyze infertility-associated biomarkers in order to assess the risk of an offspring developing a disorder. The invention recognizes that the exemplified genes may give rise to infertility issues while also being indicative of a putative offspring developing a disorder.

In certain embodiments, the biomarker is a genetic region, gene, or RNA/protein product of a gene associated with the one carbon metabolism pathway and other pathways that effect methylation of cellular macromolecules Exemplary genes and products of those genes are described below.

Methylenetetrahydrofolate Reductase (MTHFR) In particular embodiments a variant (677C>T) in the MTHFR gene is associated with infertility. The enzyme 5,10-methylenetetrahydrofolate reductase regulates folate activity (Pavlik et al., Fertility and Sterility 95(7): 2257-2262, 2011). The 677TT genotype is known in the art to be associated with 60% reduced enzyme activity, inefficient folate metabolism, decreased blood folate, elevated plasma homocysteine levels, and reduced methylation capacity. Pavlik et al. (2011) investigated the effect of the MTHFR 677C>T on serum anti-Mullerian hormone (AMH) concentrations and on the numbers of oocytes retrieved (NOR) following controlled ovarian hyperstimulation (COH). Two hundred and seventy women undergoing COH for IVF were analyzed, and their AMH levels were determined from blood samples collected after 10 days of GnRH superagonist treatment and before COH. Average AMH levels of TT carriers were significantly higher than those of homozygous CC or heterozygous CT individuals. AMH serum concentrations correlated significantly with the NOR in all individuals studied. The study concluded that the MTHFR 677TT genotype is associated with higher serum AMH concentrations but paradoxically has a negative effect on NOR after COH. It was proposed that follicle maturation might be retarded in MTHFR 677TT individuals, which could subsequently lead to a higher proportion of initially recruited follicles that produce AMH, but fail to progress towards cyclic recruitment. The tissue gene expression patterns of MTHFR do not show any bias towards oocyte expression. Analyzing a maternal sample for this variant or other variants (Table 2) in the MTHFR gene or abnormal gene expression of products of the MTHFR gene allows one to assess a risk of an offspring developing a disorder.

Jeddi-Tehrani et al. (American Journal of Reproductive Immunology 66(2):149-156, 2011) investigated the effect of the MTHFR 677TT genotype on Recurrant Pregnancy Loss (RPL). One hundred women below 35 years of age with two successive pregnancy losses and one hundred healthy women with at least two normal pregnancies were used to assess the frequency of five candidate genetic risk factors for RPL-MTHFR 677C>T, MTHFR 1298A>C, PAI1-675 4G/5G (Plasminogen Activator Inhibitor-1 promoter region), BF-455G/A (Beta Fibrinogen promoter region), and ITGB3 1565T/C (Integrin Beta 3). The frequencies of the polymorphisms were calculated and compared between case and control groups. Both the MTHFR polymorphisms (677C>T and 1298 A>C) and the BF-455G/A polymorphism were found to be positively and ITGB3 1565T/C polymorphism was found to be negatively associated with RPL. Homozygosity but not heterozygosity for the PAI-1-6754G/5G polymorphism was significantly higher in patients with RPL than in the control group. The presence of both variants in MTHFR gene highly increased the risk of RPL. Analyzing a maternal sample for these variants and other variants (Table 2) in the MTHFR gene or abnormal gene expression of products of the MTHFR gene allows one to assess a risk of an offspring developing a disorder.

Catechol-O-methyltransferase (COMT) In particular embodiments a variant (472G>A) in the COMT gene is associated with infertility. Catechol-O-methyltransferase is known in the art to be one of several enzymes that inactivates catecholamine neurotransmitters by transferring a methyl group from SAM (S-adenosyl methionine) to the catecholamine. The AA gene variant is known to alter the enzyme's thermostability and reduces its activity 3 to 4 fold (Schmidt et al., Epidemiology 22(4): 476-485, 2011). Salih et al. (Fertility and Sterility 89(5, Supplement 1): 1414-1421, 2008) investigated the regulation of COMT expression in granulosa cells and assessed the effects of 2-ME2 (COMT product) and COMT inhibitors on DNA proliferation and steroidogenesis in JC410 porcine and HGL5 human granulosa cell lines in in vitro experiments. They further assessed the regulation of COMT expression by DHT (Dihydrotestosterone), insulin, and ATRA (all-trans retinoic acid). They concluded that COMT expression in granulosa cells was up-regulated by insulin, DHT, and ATRA. Further, 2-ME2 decreased, and COMT inhibition increased granulosa cell proliferation and steroidogenesis. It was hypothesized that COMT overexpression with subsequent increased level of 2-ME2 may lead to ovulatory dysfunction. Analyzing a maternal sample for this variant in the COMT gene or abnormal gene expression of products of the COMT gene allows one to assess a risk of an offspring developing a disorder.

Methionine Synthase Reductase (MTRR) In particular embodiments a variant (A66G) in the Methionine Synthase Reductase (MTRR) gene is associated with infertility. MTRR is required for the proper function of the enzyme Methionine Synthase (MTR). MTR converts homocysteine to methionine, and MTRR activates MTR, thereby regulating levels of homocysteine and methionine. The maternal variant A66G has been associated with early developmental disorders such as Down's syndrome (Pozzi et al., 2009) and Spina Bifida (Doolin et al., American journal of human genetics 71(5): 1222-1226, 2002). Analyzing a maternal sample for this variant in the MTRR gene or abnormal gene expression of products of the MTRR gene allows one to assess a risk of an offspring developing a disorder.

Betaine-Homocysteine S-Methyltransferase (BHMT) In particular embodiments a variant (G716A) in the BHMT gene is associated with infertility. Betaine-Homocysteine S-Methyltransferase (BHMT), along with MTRR, assists in the Folate/B-12 dependent and choline/betaine-dependent conversions of homocysteine to methionine. High homocysteine levels have been linked to female infertility (Berker et al., Human Reproduction 24(9): 2293-2302, 2009). Benkhalifa et al. (2010) discuss that controlled ovarian hyperstimulation (COH) affects homocysteine concentration in follicular fluid. Using germinal vescicle oocytes from patients involved in IVF procedures, the study concludes that the human oocyte is able to regulate its homocysteine level via remethylation using MTR and BHMT, but not CBS (Cystathione Beta Synthase). They further emphasize that this may regulate the risk of imprinting problems during IVF procedures. Analyzing a maternal sample for this variant in the BHMT gene or abnormal gene expression of products of the BHMT gene allows one to assess a risk of an offspring developing a disorder.

Ikeda et al. (Journal of Experimental Zoology Part A: Ecological Genetics and Physiology 313A(3): 129-136, 2010) examined the expression patterns of all methylation pathway enzymes in bovine oocytes and preimplantation embryos. Bovine oocytes were demonstrated to have the mRNA of MAT1A (Methionine adenosyltransferase), MAT2A, MAT2B, AHCY (S-adenosylhomocysteine hydrolase), MTR, BHMT, SHMT1 (Serine hydroxymethyltransferase), SHMT2, and MTHFR. All these transcripts were consistently expressed through all the developmental stages, except MAT1A, which was not detected from the 8-cell stage onward, and BHMT, which was not detected in the 8-cell stage. Furthermore, the effect of exogenous homocysteine on preimplantation development of bovine embryos was investigated in vitro. High concentrations of homocysteine induced hypermethylation of genomic DNA as well as developmental retardation in bovine embryos. Analyzing a maternal sample for these irregular methylation patterns allows one to assess a risk of an offspring developing a disorder.

Folate Receptor 2 (FOLR2) In particular embodiments a variant (rs2298444) in the FOLR2 gene is associated with infertility. Folate Receptor 2 helps transport folate (and folate derivatives) into cells. Elnakat and Ratnam (Frontiers in bioscience: a journal and virtual library 11: 506-519, 2006) implicate FOLR2, along with FOLR1, in ovarian and endometrial cancers. Analyzing a maternal sample for variants in the FOLR2 or FOLR1 genes or abnormal gene expression of products of the FOLR2 or FOLR1 genes allows one to assess a risk of an offspring presenting with a developmental disorder.

Transcobalamin 2 (TCN2) In particular embodiments a variant (C776G) in the TCN2 gene is associated with infertility. Transcobalamin 2 facilitates transport of cobalamin (Vitamin B12) into cells. Stanislawska-Sachadyn et al. (Eur J ClinNutr 64(11): 1338-1343, 2010) assessed the relationship between TCN2 776C>G polymorphism and both serum B12 and total homocysteine (tHcy) levels. Genotypes from 613 men from Northern Ireland were used to show that the TCN2 776CC genotype was associated with lower serum B12 concentrations when compared to the 776CG and 776GG genotypes. Furthermore, vitamin B12 status was shown to influence the relationship between TCN2 776C>G genotype and tHcy concentrations. The TCN2 776C>G polymorphism may contribute to the risk of pathologies associated with low B12 and high total homocysteine phenotype. Analyzing a maternal sample for this variant in the TCN2 gene or abnormal gene expression of products of the TCN2 gene allows one to assess a risk of an offspring developing a disorder.

Cystathionine-Beta-Synthase (CBS) In particular embodiments a variant (rs234715) in the CBS gene is associated with infertility. With vitamin B6 as a cofactor, the Cystathionine-Beta-Synthase (CBS) enzyme catalyzes a reaction that permanently removes homocysteine from the methionine pathway by diverting it to the transsulfuration pathway. CBS gene variants associated with decreased CBS activity also lead to elevated plasma homocysteine levels. Guzman et al. (2006) demonstrate that Cbs knockout mice are infertile. They further explain that Cbs-null female infertility is a consequence of uterine failure, which is a consequence of hyperhomocysteinemia or other factor(s) in the uterine environment. Analyzing a maternal sample for this variant in the CBS gene or abnormal gene expression of products of the CBS gene allows one to assess a risk of an offspring presenting with a developmental disorder.

DNA (cytosine-5)-methyltransferase 1 (DNMT1) In particular embodiments a variant (rs16999593) in the DNMT1 gene is associated with infertility. We identified the rs16999593 variant, which causes a histidine (H) to arginine (R) change at residue 97 in Dnmtl protein, in a subset of infertile female patients. There are two isoforms of DNMT1 protein that are expressed in a sequential order during development (Carlson et al., 1992). The DNMT1o protein is known in mice to be a maternal effect protein that is synthesized in the oocyte and functions after fertilization to maintain methylation patterns on imprinted alleles. The DNMT1 protein, which has the same primary structure as DNMT1o with an additional 118-aa domain at its amino terminus, replaces DNMT1o after embryo implantation. Both DNMT1 isoforms maintain methylation at CpG dinucleotides by catalyzing the addition of methyl groups to cytosine bases in DNA, and are implicated in stabilizing repeat sequences, including CAG repeats.

Aberrant expression or function of both DNMT1 and/or DNMT1o could lead to female infertility and the presentation of disorders in putative offspring if infertility is bypassed. Depletion of DNMT1 in mice leads to an increase in the expansion of CAG repeats during transmission from parents to offspring (Dion et al., 2008). Furthermore, a reduction of DNMT1 causes mismatch repair defects (Loughery et al., 2011) and destabilizes CAG triplet repeats (Dion et al., 2008) in human cells. Expanded CAG repeat tracks can cause an assortment of neurodegenerative diseases, including, but not limited to Huntington disease (HD), spino-cerebellar ataxia 1, 2, 3, 6, 7 and 17 (SCA1/2/3/6/7/17), and dentatorubral-pallidoluysian atrophy (DRPLA). Female mice with a homozygous Dnmtlo deletion appear phenotypically normal, but heterozygous fetuses of homozygous females generally die during the last 3^{rd} of gestation due to loss of allele-specific gene expression and methylation at certain imprinted loci{Howell:2001hf}. The rs16999593 variant in DNMT1 could alter or attenuate DNMT1/DNMT1o function, which would subsequently lead to intergenerational CAG repeat track expansion and/or loss of imprinting, both of which could increase the risk of an offspring disorder. Analyzing a maternal sample for this or other variants in the DNMT1 gene or abnormal gene expression of products of the DNMT1 gene allows one to assess a risk of an offspring developing a disorder.

In certain embodiments, the biomarker is a genetic region that has been previously associated with female infertility. A SNP association study by targeted re-sequencing was performed to search for new genetic variants associated with female infertility. Such methods have been successful in identifying significant variants associated in a wide range of diseases Rehman et al., 2010; Walsh et al., 2010). Briefly, a SNP association study is performed by collecting SNPs in genetic regions of interest in a number of samples and controls and then testing each of the SNPs that showed significant frequency differences between cases and controls. Significant frequency differences between cases and controls indicate that the SNP is associated with the condition of interest.

Table 2 identifies 286 SNPs associated with female infertility that fall within genetic regions that have also been associated with the risk of an offspring developing a disorder. In particular embodiments, the infertility-associated genetic region is selected from the SNPs shown in Table 2 below.

TABLE 2
Infertility-Associated SNPs

| SNP ID | Locus | Position | Ref | Var | MAF | Score |
|---|---|---|---|---|---|---|
| novel | MUC4 | chr3: 195512107 | T | A | — | 96.00 |
| rs62532306 | BOP1 | chr8: 145505229 | C | A | — | 58.06 |
| rs4002465 | CGB | chr19: 49527061 | G | C | — | 54.04 |
| rs62126025 | CGB | chr19: 49527066 | T | C | — | 54.04 |
| rs76295320 | ZNF787 | chr19: 56610009 | A | G | — | 53.81 |
| rs62126988 | RFPL4A | chr19: 56272921 | A | T | — | 49.48 |
| rs3972768 | ZP3 | chr7: 76063333 | T | C | — | 47.94 |
| rs9915824 | HSD17B1 | chr17: 40702841 | A | G | — | 47.74 |
| rs74842155 | RFPL4A | chr19: 56273736 | T | A | — | 46.46 |
| rs74182589 | ZNF787 | chr19: 56610006 | T | C | — | 46.07 |
| rs79852613 | ZNF787 | chr19: 56610038 | T | C | — | 46.07 |
| rs55956596 | MAD1L1 | chr7: 1922620 | C | T | — | 45.89 |
| rs112808291 | MUC4 | chr3: 195507242 | C | A | — | 44.66 |
| rs10835907 | WT1 | chr11: 32434180 | G | A | — | 43.48 |
| rs2688498 | MUC4 | chr3: 195476442 | G | C | — | 42.95 |
| rs28571120 | CGB | chr19: 49526191 | G | A | — | 42.93 |
| rs116383373 | RFPL4A | chr19: 56268106 | C | T | — | 41.07 |
| rs111519111 | MAD1L1 | chr7: 1910705 | T | C | — | 41.07 |
| novel | ZNF787 | chr19: 56610083 | T | C | — | 40.52 |
| rs13406100 | BARD1 | chr2: 215627612 | C | A | — | 38.66 |
| rs10860866 | IGF1 | chr12: 102835687 | T | G | — | 38.14 |
| rs2868052 | RFPL4A | chr19: 56276194 | A | C | — | 38.14 |
| rs2450440 | ZNF787 | chr19: 56628695 | A | G | — | 38.14 |
| rs17852109 | CGB | chr19: 49526203 | G | C | — | 38.12 |
| rs1544809 | MUC4 | chr3: 195472740 | C | T | — | 37.66 |
| rs59080007 | CARM1 | chr19: 10996568 | T | C | — | 37.43 |
| rs7633103 | MUC4 | chr3: 195471492 | C | G | — | 37.21 |
| rs62456177 | PMS2 | chr7: 6025845 | C | T | — | 36.82 |
| rs1638149 | ZP3 | chr7: 76064950 | A | C | — | 36.25 |
| rs1754208 | ZP3 | chr7: 76066129 | A | G | — | 36.25 |
| rs4386875 | MAD1L1 | chr7: 1928020 | G | A | — | 35.26 |
| rs12973299 | ZNF787 | chr19: 56618129 | C | T | — | 34.40 |
| novel | MUC4 | chr3: 195507846 | C | G | — | 33.33 |
| novel | MTO1 | chr6: 74206880 | C | T | — | 33.33 |
| rs6969822 | MAD1L1 | chr7: 2035814 | G | C | — | 33.33 |
| rs2045865 | RFPL4A | chr19: 56273902 | C | A | — | 32.02 |
| novel | HS6ST1 | chr2: 129006024 | C | A | — | 31.99 |
| rs12403346 | FMN2 | chr1: 240620371 | G | A | — | 31.64 |
| rs113457754 | MUC4 | chr3: 195512343 | G | A | — | 30.93 |
| rs1770345 | MTOR | chr1: 11214580 | C | A | — | 30.47 |
| rs72626227 | CGB | chr19: 49527339 | C | T | — | 30.47 |
| rs2948677 | MUC4 | chr3: 195510786 | T | G | — | 30.47 |
| rs413807 | MUC4 | chr3: 195510827 | C | T | — | 30.47 |
| rs71321841 | MUC4 | chr3: 195513345 | A | C | — | 30.47 |
| rs67816107 | ESRRB | chr14: 76944784 | T | C | — | 30.04 |
| rs2019946 | RFPL4A | chr19: 56273293 | G | A | — | 30.04 |
| novel | EZR | chr6: 159237011 | A | C | — | 29.57 |
| rs9283487 | PRKRA | chr2: 179296271 | T | C | — | 29.28 |
| rs112638514 | MUC4 | chr3: 195539373 | T | C | — | 29.02 |
| novel | UIMC1 | chr5: 176396948 | A | C | — | 29.02 |
| rs3997878 | PRKRA | chr2: 179296626 | G | A | — | 28.93 |
| rs71523271 | MAD1L1 | chr7: 1910374 | G | A | — | 28.93 |
| rs9881716 | MUC4 | chr3: 195472148 | C | T | — | 28.52 |
| novel | MUC4 | chr3: 195506267 | C | T | — | 28.52 |
| rs5022939 | MAD1L1 | chr7: 2035966 | G | C | — | 28.50 |
| novel | ZP3 | chr7: 76072644 | A | G | — | 28.50 |
| novel | ZNF787 | chr19: 56610075 | C | A | — | 28.07 |
| rs78772566 | ZNF787 | chr19: 56610076 | A | G | — | 28.07 |
| rs62475576 | ZP3 | chr7: 76073794 | A | T | — | 28.07 |
| novel | COMT | chr22: 19947360 | C | T | — | 27.78 |
| novel | COMT | chr22: 19947361 | A | T | — | 27.78 |
| rs10408967 | CGB | chr19: 49528275 | C | T | — | 27.65 |
| novel | MUC4 | chr3: 195507827 | G | A | — | 27.65 |
| novel | MTO1 | chr6: 74192512 | C | T | — | 27.16 |
| novel | GPC3 | chrX: 132910905 | C | T | — | 27.16 |
| novel | ESR1 | chr6: 152306694 | G | A | — | 26.61 |
| novel | STK3 | chr8: 99891810 | A | C | — | 26.61 |
| rs3997877 | PRKRA | chr2: 179296666 | T | C | — | 26.58 |
| rs3997879 | PRKRA | chr2: 179296511 | C | T | — | 26.26 |
| rs58599527 | MUC4 | chr3: 195476305 | G | A | — | 26.12 |
| rs79313055 | ZP3 | chr7: 76073796 | G | A | — | 26.12 |
| novel | ZNF787 | chr19: 56610073 | A | G | — | 25.67 |
| rs78006633 | MUC4 | chr3: 195481809 | A | G | — | 24.90 |
| novel | MUC4 | chr3: 195481867 | A | G | — | 24.90 |
| novel | NLRP11 | chr19: 56312625 | G | T | — | 24.74 |
| novel | ESR1 | chr6: 152201316 | G | T | — | 24.74 |
| novel | MAD1L1 | chr7: 2264910 | A | G | — | 24.55 |
| novel | MUC4 | chr3: 195508709 | A | G | — | 24.19 |
| rs79018306 | HSD17B1 | chr17: 40708178 | C | T | — | 23.93 |
| novel | FMR1 | chrX: 147021719 | G | A | — | 23.71 |
| novel | DIAPH2 | chrX: 96726717 | G | A | — | 23.71 |
| novel | CGB5 | chr19: 49546958 | T | C | — | 23.28 |
| novel | CGB5 | chr19: 49546960 | T | A | — | 23.28 |
| novel | MUC4 | chr3: 195499065 | G | A | — | 23.28 |
| novel | MUC4 | chr3: 195508500 | G | C | — | 23.28 |
| rs12278181 | HSD17B12 | chr11: 43704110 | A | C | — | 22.89 |
| novel | CTCF | chr16: 67640652 | G | T | — | 22.89 |
| novel | NLRP5 | chr19: 56542077 | C | T | — | 22.89 |
| rs2906996 | ZP3 | chr7: 76069937 | C | T | — | 22.89 |
| rs437805 | MUC4 | chr3: 195512103 | G | A | — | 22.53 |
| novel | MTO1 | chr6: 74192508 | C | T | — | 22.32 |
| novel | MAD1L1 | chr7: 2042637 | C | T | — | 22.32 |
| novel | MAD1L1 | chr7: 2042638 | T | C | — | 22.32 |
| rs34718891 | CBS | chr21: 44477834 | C | G | — | 22.20 |
| novel | ZNF787 | chr19: 56618184 | G | T | — | 21.89 |
| novel | MAD1L1 | chr7: 1898732 | C | G | — | 21.89 |
| novel | EIF2B2 | chr14: 75465532 | T | A | — | 21.78 |
| rs12489838 | MUC4 | chr3: 195514109 | C | A | — | 21.78 |
| rs76816122 | MUC4 | chr3: 195514144 | T | C | — | 21.78 |
| novel | NLRP11 | chr19: 56332403 | G | T | — | 21.31 |
| rs7621695 | MUC4 | chr3: 195475989 | G | A | — | 21.08 |
| rs112829487 | MUC4 | chr3: 195539376 | G | A | — | 20.89 |
| novel | EZR | chr6: 159232393 | A | T | — | 20.89 |
| rs61762234 | TP73 | chr1: 3610404 | A | C | — | 20.52 |
| novel | HS6ST1 | chr2: 129024942 | C | G | — | 20.51 |
| novel | MUC4 | chr3: 195514728 | T | A | — | 20.51 |
| novel | MUC4 | chr3: 195481823 | G | A | — | 20.17 |
| novel | TP73 | chr1: 3610392 | G | A | — | 19.89 |
| novel | STK3 | chr8: 99693203 | A | C | — | 19.89 |
| novel | DIAPH2 | chrX: 96444382 | C | A | — | 19.89 |
| rs62126040 | CGB2 | chr19: 49539560 | T | C | — | 19.85 |
| rs13046562 | CBS | chr21: 44477803 | C | G | — | 19.85 |
| novel | MUC4 | chr3: 195507262 | T | G | — | 19.56 |
| rs71522278 | MAD1L1 | chr7: 1943503 | C | T | — | 19.56 |
| rs10950559 | MAD1L1 | chr7: 2172705 | C | T | — | 19.56 |
| novel | ESRRB | chr14: 76929788 | G | T | — | 19.36 |
| novel | TLE6 | chr19: 2993943 | T | G | — | 19.36 |
| novel | GALT | chr9: 34637819 | G | C | — | 19.36 |
| rs3176202 | POLG | chr15: 89866286 | G | A | — | 18.80 |
| rs798767 | TACC3 | chr4: 1734281 | A | G | — | 18.58 |
| novel | MDM4 | chr1: 204521995 | C | T | — | 18.50 |
| novel | GDF1 | chr19: 18985313 | T | G | — | 18.14 |
| rs113643133 | ZNF787 | chr19: 56609885 | A | G | — | 17.81 |
| novel | ZNF787 | chr19: 56625688 | T | C | — | 17.81 |
| novel | TP73 | chr1: 3585751 | G | C | — | 17.47 |
| novel | ESRRB | chr14: 76936269 | G | T | — | 17.47 |
| novel | ATR | chr3: 142282826 | A | C | — | 17.47 |
| novel | BRCA1 | chr17: 41264749 | C | G | — | 17.24 |
| rs12539440 | MAD1L1 | chr7: 1980610 | C | G | — | 16.95 |
| rs7936739 | NLRP14 | chr11: 7068543 | C | T | — | 16.75 |
| novel | MAD2L1BP | chr6: 43605162 | C | T | — | 16.50 |
| novel | DIAPH2 | chrX: 96149969 | C | T | — | 16.50 |
| rs55976062 | TP73 | chr1: 3622892 | T | C | — | 14.93 |
| rs62282465 | MUC4 | chr3: 195506315 | T | C | — | 14.93 |
| rs55782426 | PMS2 | chr7: 6023033 | T | C | — | 14.70 |
| rs6802709 | MUC4 | chr3: 195473776 | T | C | — | 14.48 |
| rs6768267 | MUC4 | chr3: 195473783 | G | A | — | 14.48 |
| rs10271093 | MAD1L1 | chr7: 1885221 | C | T | — | 14.48 |
| rs1558180 | ZP3 | chr7: 76066189 | A | G | — | 14.48 |
| rs9835456 | MUC4 | chr3: 195471522 | G | A | — | 13.74 |
| rs13239578 | MAD1L1 | chr7: 1980636 | G | C | — | 13.42 |
| rs80209248 | ZP3 | chr7: 76069486 | G | A | — | 12.65 |
| rs61124766 | NLRP5 | chr19: 56511465 | T | C | — | 12.43 |
| rs7251474 | NLRP7 | chr19: 55443228 | A | T | — | 12.24 |
| rs12155464 | MAD1L1 | chr7: 1899547 | G | C | — | 12.24 |
| rs12155227 | MAD1L1 | chr7: 1899553 | T | C | — | 12.24 |
| rs80123784 | MUC4 | chr3: 195513383 | T | A | — | 12.05 |
| rs75239380 | RFPL4A | chr19: 56274453 | G | A | — | 11.88 |
| rs578069 | ZNF787 | chr19: 56631356 | A | G | — | 11.88 |
| rs75200017 | MUC4 | chr3: 195473784 | C | T | — | 11.43 |
| rs2880893 | MUC4 | chr3: 195491180 | C | T | — | 11.07 |
| rs10408095 | CGB | chr19: 49527991 | A | G | — | 10.82 |
| rs10408850 | CGB | chr19: 49528047 | G | A | — | 10.82 |
| rs35280967 | MAD1L1 | chr7: 1922667 | C | G | — | 10.16 |
| rs55740147 | MAD1L1 | chr7: 2089773 | G | A | — | 9.71 |
| rs62075836 | HSD17B1 | chr17: 40702252 | C | T | — | 9.43 |
| rs4662595 | HS6ST1 | chr2: 129024915 | C | T | — | 9.00 |
| rs71635066 | MUC4 | chr3: 195477586 | T | C | — | 9.00 |
| rs80197731 | PRKRA | chr2: 179315890 | G | C | — | 8.74 |
| rs34406439 | TP73 | chr1: 3610322 | A | G | — | 8.52 |
| rs11757217 | FOXO3 | chr6: 108882570 | C | T | — | 8.32 |
| rs78190054 | TP73 | chr1: 3610357 | G | A | — | 8.14 |
| rs13123646 | TACC3 | chr4: 1740539 | T | C | — | 8.14 |
| rs60234611 | MAD1L1 | chr7: 1885242 | T | C | — | 7.98 |
| rs7257437 | NLRP5 | chr19: 56532128 | T | C | — | 7.70 |
| rs4719408 | MAD1L1 | chr7: 2089757 | T | C | — | 7.70 |
| rs62284989 | MUC4 | chr3: 195479484 | G | A | — | 7.57 |
| rs113971556 | BOP1 | chr8: 145505306 | A | C | — | 7.45 |
| rs13078480 | MUC4 | chr3: 195499099 | C | T | — | 7.14 |
| rs77907507 | MUC4 | chr3: 195514104 | T | A | — | 6.66 |
| rs112413534 | ZNF787 | chr19: 56603294 | A | C | — | 6.44 |
| rs112012272 | ZNF787 | chr19: 56625720 | T | C | — | 5.95 |
| rs71355791 | PLA2G4C | chr19: 48564911 | T | C | 0.01 | 5.86 |
| rs4801657 | NLRP5 | chr19: 56515593 | C | T | — | 5.82 |
| rs55846169 | MAD1L1 | chr7: 2089802 | C | T | — | 5.82 |
| rs113631808 | PMS2 | chr7: 6019274 | C | T | — | 5.70 |
| rs115280443 | HADHA | chr2: 26451922 | A | G | 0.02 | 5.57 |
| rs10407868 | CGB | chr19: 49527907 | A | C | — | 5.22 |
| rs34349826 | LHB | chr19: 49519883 | A | G | — | 5.09 |
| rs79014552 | TP63 | chr3: 189499326 | G | T | 0.01 | 5.04 |
| rs117469552 | FMN2 | chr1: 240525487 | C | T | 0.00 | 4.83 |
| rs77453381 | TP73 | chr1: 3610436 | C | G | — | 4.83 |
| rs11667729 | ZNF787 | chr19: 56628807 | T | C | — | 4.83 |
| rs11806926 | FMN2 | chr1: 240569344 | C | G | 0.05 | 4.78 |
| rs79723785 | BRSK1 | chr19: 55818225 | T | C | 0.00 | 4.73 |
| rs75739598 | HADHA | chr2: 26412802 | A | G | 0.02 | 4.73 |
| rs78522745 | NLRP8 | chr19: 56461003 | G | T | 0.00 | 4.67 |
| rs79288206 | RFPL4A | chr19: 56276174 | G | A | — | 4.55 |
| rs2637100 | ZNF787 | chr19: 56628814 | C | G | — | 4.55 |
| rs2016745 | AFF2 | chrX: 147628728 | A | T | — | 4.55 |
| rs56034002 | AFF2 | chrX: 147628775 | A | T | — | 4.55 |
| rs73102793 | ACVR1B | chr12: 52390574 | C | T | 0.02 | 4.52 |
| rs11798175 | DIAPH2 | chrX: 96741405 | G | A | 0.06 | 4.46 |
| rs12145827 | FMN2 | chr1: 240596675 | C | T | 0.36 | 4.35 |
| rs67341807 | PADI6 | chr1: 17710800 | A | G | 0.04 | 4.34 |
| rs12025170 | PADI6 | chr1: 17704934 | G | C | 0.14 | 4.29 |
| rs3800869 | MAD1L1 | chr7: 2109933 | C | T | 0.24 | 4.22 |
| rs55765849 | ZNF787 | chr19: 56603305 | G | A | 0.24 | 4.22 |
| rs1204891 | PADI6, PADI4 | chr1: 17694615 | G | A | 0.18 | 4.18 |
| rs1464541 | MAD1L1 | chr7: 1960887 | A | G | 0.34 | 4.14 |
| rs34647879 | MAD1L1 | chr7: 1960646 | G | A | 0.34 | 4.14 |
| rs74392263 | BMP7 | chr20: 55775929 | A | G | 0.05 | 4.13 |
| rs75100250 | TAF4B | chr18: 23814766 | C | T | 0.05 | 4.09 |
| rs34008721 | MAD1L1 | chr7: 1959975 | T | C | 0.35 | 4.08 |
| rs1107238 | HADHA | chr2: 26458244 | T | C | 0.02 | 4.07 |
| rs74406635 | ZNF787 | chr19: 56603338 | G | A | — | 4.04 |
| rs17627983 | EZH2 | chr7: 148528058 | A | G | 0.08 | 4.00 |
| rs116832355 | FMN2 | chr1: 240299858 | T | A | 0.01 | 3.99 |
| rs10265212 | MAD1L1 | chr7: 1860733 | G | C | — | 3.92 |
| rs78501539 | TP63 | chr3: 189431642 | A | G | 0.07 | 3.91 |
| rs55815720 | LIFR | chr5: 38588247 | C | A | 0.33 | 3.90 |
| rs72912353 | SMAD2 | chr18: 45369316 | T | C | 0.06 | 3.89 |
| rs12563231 | PADI6 | chr1: 17703861 | C | T | 0.14 | 3.85 |
| rs117748025 | MYC | chr8: 128747011 | C | A | 0.05 | 3.85 |
| rs76633823 | ZNF787 | chr19: 56610026 | A | G | 0.33 | 3.85 |
| rs75288315 | EZH2 | chr7: 148530212 | T | C | 0.08 | 3.84 |
| rs4618595 | MAD1L1 | chr7: 1946106 | A | G | 0.31 | 3.82 |
| rs2631635 | ZNF787 | chr19: 56628705 | G | A | 0.20 | 3.79 |
| rs10497192 | ACVR1 | chr2: 158671700 | C | T | 0.49 | 3.78 |
| rs9812619 | TP63 | chr3: 189391823 | C | A | 0.31 | 3.77 |
| rs4721162 | MAD1L1 | chr7: 1930350 | A | G | 0.29 | 3.76 |
| rs113242110 | ZNF787 | chr19: 56609882 | T | C | 0.06 | 3.75 |
| rs1671187 | NLRP2, NLRP7 | chr19: 55476685 | A | T | — | 3.73 |
| rs11571700 | BRCA2 | chr13: 32927017 | G | A | 0.05 | 3.71 |
| rs77497094 | KHDC1 | chr6: 73958003 | T | C | 0.00 | 3.69 |
| rs6978209 | MAD1L1 | chr7: 1931544 | G | A | 0.32 | 3.68 |
| rs73173010 | BRCA2 | chr13: 32975041 | G | A | 0.05 | 3.68 |
| rs2280549 | MAD1L1 | chr7: 1976635 | G | A | 0.26 | 3.65 |
| rs511297 | ZNF787 | chr19: 56609944 | C | T | — | 3.64 |
| rs57766906 | ZP3 | chr7: 76064351 | G | A | — | 3.64 |
| rs3812043 | LIFR | chr5: 38597478 | A | G | 0.32 | 3.63 |
| rs66617818 | MAD1L1 | chr7: 1872132 | C | A | 0.44 | 3.62 |
| rs564533 | C6orf221 | chr6: 74072937 | G | C | 0.07 | 3.60 |
| rs76955461 | FMN2 | chr1: 240565203 | C | T | 0.01 | 3.57 |
| rs117986556 | TP63 | chr3: 189505273 | G | T | 0.00 | 3.57 |
| rs7616592 | MUC4 | chr3: 195476380 | A | G | — | 3.57 |
| rs10802871 | FMN2 | chr1: 240613707 | G | A | 0.24 | 3.56 |
| rs28572042 | NLRP7 | chr19: 55447341 | T | C | 0.06 | 3.55 |
| rs28617363 | NLRP7 | chr19: 55447342 | C | T | 0.06 | 3.55 |
| rs111468036 | ESR1 | chr6: 152418902 | C | T | 0.00 | 3.53 |
| rs12733042 | GREM2 | chr1: 240719255 | T | C | 0.15 | 3.53 |
| rs11739017 | LIFR | chr5: 38519462 | T | C | 0.29 | 3.52 |
| rs75584969 | TFPI | chr2: 188388402 | G | C | 0.04 | 3.51 |
| rs12859009 | DIAPH2 | chrX: 96707326 | C | T | 0.40 | 3.50 |
| rs73173011 | BRCA2 | chr13: 32975487 | T | C | 0.05 | 3.50 |
| rs60506574 | CGB2 | chr19: 49534393 | T | C | — | 3.50 |
| rs6641437 | AFF2 | chrX: 147660644 | C | T | 0.17 | 3.49 |
| rs2228948 | ACVR1 | chr2: 158593905 | C | T | 0.13 | 3.49 |
| rs7810386 | MAD1L1 | chr7: 1952031 | T | A | 0.32 | 3.48 |
| rs7535085 | FMN2 | chr1: 240183908 | C | T | 0.18 | 3.48 |
| rs80009068 | FMN2 | chr1: 240554884 | T | G | 0.02 | 3.48 |
| rs12760504 | PADI6 | chr1: 17696247 | T | C | 0.20 | 3.47 |
| rs12880959 | ESRRB | chr14: 76787228 | C | T | 0.40 | 3.46 |
| rs75301713 | ESR1 | chr6: 152295764 | C | G | 0.01 | 3.46 |
| rs74501504 | ESR1 | chr6: 152306334 | C | T | 0.01 | 3.46 |
| rs72914334 | SMAD2 | chr18: 45452084 | C | T | 0.02 | 3.46 |
| rs6700319 | PADI6 | chr 1: 17698891 | C | T | 0.20 | 3.45 |
| rs7785626 | MAD1L1 | chr7: 2103005 | A | G | 0.28 | 3.45 |
| rs10274701 | EZH2 | chr7: 148552456 | C | T | 0.24 | 3.44 |
| rs511291 | ZNF787 | chr19: 56609952 | A | G | — | 3.43 |
| rs12859787 | DIAPH2 | chrX: 96658300 | A | G | 0.40 | 3.43 |
| rs77404600 | GREM2 | chr1: 240754703 | G | A | 0.00 | 3.42 |
| rs12886462 | ESRRB | chr14: 76793400 | T | G | 0.40 | 3.39 |
| rs73945411 | CGB5 | chr19: 49542070 | G | C | — | 3.37 |
| rs314287 | LIN28B | chr6: 105434661 | A | G | 0.43 | 3.36 |
| rs6429209 | GREM2 | chr1: 240707877 | C | G | 0.46 | 3.35 |
| rs3748697 | CENPF | chr1: 214820099 | A | G | 0.27 | 3.33 |
| rs17318323 | AFF2 | chrX: 148079412 | A | G | 0.03 | 3.33 |
| rs2067948 | MAD1L1 | chr7: 1976749 | G | A | 0.28 | 3.33 |
| rs2072408 | EZH2 | chr7: 148508197 | A | G | 0.25 | 3.32 |
| rs12132059 | FMN2 | chr1: 240588861 | A | T | 0.00 | 3.31 |
| rs6944660 | MAD1L1 | chr7: 2083782 | A | G | 0.27 | 3.31 |
| rs17646552 | NLRP5 | chr19: 56565263 | T | C | 0.00 | 3.28 |
| rs10512687 | LIFR | chr5: 38581262 | C | T | 0.33 | 3.28 |
| rs269955 | NLRP7 | chr19: 55443424 | A | G | 0.28 | 3.28 |
| rs12699596 | MAD1L1 | chr7: 2065828 | A | G | 0.25 | 3.27 |
| rs5921851 | DIAPH2 | chrX: 96811308 | T | A | 0.41 | 3.27 |
| rs385564 | KL | chr13: 33592409 | C | G | 0.31 | 3.27 |
| rs10853881 | NLRP4 | chr19: 56373608 | A | G | 0.28 | 3.25 |
| rs2561821 | LIFR | chr5: 38488174 | C | A | 0.43 | 3.24 |
| rs12760851 | FMN2 | chr1: 240569361 | T | G | 0.02 | 3.22 |
| rs8105736 | TLE6 | chr19: 2973274 | C | T | 0.01 | 3.22 |
| rs17445954 | TP63 | chr3: 189411830 | G | A | 0.10 | 3.21 |
| rs12735781 | FMN2 | chr1: 240569335 | G | C | 0.10 | 3.21 |
| rs1204889 | PADI6, PADI4 | chr1: 17694717 | T | A | 0.22 | 3.21 |
| rs112915167 | ESRRB | chr14: 76899709 | G | A | 0.00 | 3.21 |

The SNPs listed in the Table independently and significantly associated with Female Infertility. Position refers to NCBI Build 37. Alleles are reported on the forward strand.
Ref=Reference allele,
Var=Variant (risk) allele,
MAF=minor allele frequency,
Score=P-value of one-sided exact binomial test-Log ₁₀p-value

In certain embodiments, the biomarker is a genetic region, gene or gene product of a gene associated with human fertility and another disorder. Examples of offspring disorders include, but are not limited to, neurodevelopmental, neuropsychological and neuro-genetic disorder, e.g. neural tube defects, an autism spectrum disorder (including, but not limited to classical autism, asperger syndrome, rett syndrome, childhood disintegrative disorder, and pervasive developmental disorder not otherwise specified (PDD-NOS)), Bardet-Beidl syndrome, Attention Deficit Hyperactivity Disorder (ADHD), Angelman Syndrome, Prader-Willi Syndrome, Bipolar Disorder, Charcot Marie Tooth Syndrome, or Schizophrenia; metabolic disorder, e.g. obesity and Diabetes Mellitus (Type I or II); gynecological and/or infertility disorder, e.g. Endometriosis and Premature ovarian failure (POF); autoimmune disorder, e.g. asthma, juvenile idiopathic arthritis, allergies, Addison's disease, Crohn's disease, and Celiac disease; muscular dystrophy; cancer; and cardiovascular disease, e.g. early onset coronary heart disease.

### Assays

Methods of the invention involve conducting an assay that detects either a variant in an infertility-associated gene or abnormal expression (over or under) of an infertility-associated gene product. In particular embodiments, the assay is conducted on infertility-associated genetic regions or products of these regions. Detailed descriptions of conventional methods, such as those employed to make and use nucleic acid arrays, amplification primers, hybridization probes, and the like can be found in standard laboratory manuals such as: Genome Analysis: A Laboratory Manual Series (Vols. I-IV), Cold Spring Harbor Laboratory Press; PCR Primer: A Laboratory Manual, Cold Spring Harbor Laboratory Press; and Sambrook, J et al., (2001) Molecular Cloning: A Laboratory Manual, 2nd ed. (Vols. 1-3), Cold Spring Harbor Laboratory Press. Custom nucleic acid arrays are commercially available from, e.g., Affymetrix (Santa Clara, Calif.), Applied Biosystems (Foster City, Calif.), and Agilent Technologies (Santa Clara, Calif.).

Methods of detecting variants in genetic regions are known in the art. In certain embodiments, a variant in a single infertility-associated genetic region indicates infertility. In other embodiments, the assay is conducted on more than one genetic region, and a variant in at least two of the genetic regions indicates infertility. In other embodiments, a variant in at least three of the genetic regions indicates infertility; a variant in at least four of the genetic regions indicates infertility; a variant in at least five of the genetic regions indicates infertility; a variant in at least six of the genetic regions indicates infertility; a variant in at least seven of the genetic regions indicates infertility; a variant in at least eight of the genetic regions indicates infertility; a variant in at least nine of the genetic regions indicates infertility; a variant in at least 10 of the genetic regions indicates infertility; a variant in at least 15 of the genetic regions indicates infertility; or a variant in all of the genetic regions from Table 1 indicates infertility.

In certain embodiments, a known single nucleotide polymorphism at a particular position can be detected by single base extension for a primer that binds to the sample DNA adjacent to that position. See for example Shuber et al. (U.S. Pat. No. 6,566,101), the content of which is incorporated by reference herein in its entirety. In other embodiments, a hybridization probe might be employed that overlaps the SNP of interest and selectively hybridizes to sample nucleic acids containing a particular nucleotide at that position. See for example Shuber et al. (U.S. Pat. Nos. 6,214,558 and 6,300,077), the content of which is incorporated by reference herein in its entirety.

In particular embodiments, nucleic acids are sequenced in order to detect variants (i.e., mutations) in the nucleic acid compared to wild-type and/or non-mutated forms of the sequence. The nucleic acid can include a plurality of nucleic acids derived from a plurality of genetic elements. Methods of detecting sequence variants are known in the art, and sequence variants can be detected by any sequencing method known in the art e.g., ensemble sequencing or single molecule sequencing.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes.

One conventional method to perform sequencing is by chain termination and gel separation, as described by Sanger et al., Proc Natl. Acad. Sci. USA, 74(12): 5463 67 (1977). Another conventional sequencing method involves chemical degradation of nucleic acid fragments. See, Maxam et al., Proc. Natl. Acad. Sci., 74: 560 564 (1977). Methods have also been developed based upon sequencing by hybridization. See, e.g., Harris et al., (U.S. patent application number 2009/0156412). The content of each reference is incorporated by reference herein in its entirety.

A sequencing technique that can be used in the methods of the provided invention includes, for example, Helicos True Single Molecule Sequencing (tSMS) (Harris T. D. et al. (2008) Science 320:106-109). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step. Further description of tSMS is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety.

Another example of a DNA sequencing technique that can be used in the methods of the provided invention is 454 sequencing (Roche) (Margulies, M et al. 2005, Nature, 437, 376-380). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used in the methods of the provided invention is SOLiD technology (Applied Biosystems). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a DNA sequencing technique that can be used in the methods of the provided invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and is attached at a resolution such that the fragments are individually resolvable. Addition of one or more nucleotides releases a proton (H⁺), which signal detected and recorded in a sequencing instrument. The signal strength is proportional to the number of nucleotides incorporated.

Another example of a sequencing technology that can be used in the methods of the provided invention is 11lumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA (for example, as described in US Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using a electron microscope (Moudrianakis E. N. and Beer M. Proc Natl Acad Sci USA. 1965 March; 53:564-71). In one example of the technique, individual DNA molecules are labeled using metallic labels that are distinguishable using an electron microscope. These molecules are then stretched on a flat surface and imaged using an electron microscope to measure sequences.

If the nucleic acid from the sample is degraded or only a minimal amount of nucleic acid can be obtained from the sample, PCR can be performed on the nucleic acid in order to obtain a sufficient amount of nucleic acid for sequencing (See e.g., Mullis et al. U.S. Pat. No. 4,683,195, the contents of which are incorporated by reference herein in its entirety).

Methods of detecting levels of gene products (e.g., RNA or protein) are known in the art. Commonly used methods known in the art for the quantification of mRNA expression in a sample include northern blotting and in situ hybridization (Parker & Barnes, Methods in Molecular Biology 106:247 283 (1999), the contents of which are incorporated by reference herein in their entirety); RNAse protection assays (Hod, Biotechniques 13:852 854 (1992), the contents of which are incorporated by reference herein in their entirety); and PCR-based methods, such as reverse transcription polymerase chain reaction (RT-PCR) (Weis et al., Trends in Genetics 8:263 264 (1992), the contents of which are incorporated by reference herein in their entirety). Alternatively, antibodies may be employed that can recognize specific duplexes, including RNA duplexes, DNA-RNA hybrid duplexes, or DNA-protein duplexes. Other methods known in the art for measuring gene expression (e.g., RNA or protein amounts) are shown in Yeatman et al. (U.S. patent application number 2006/0195269), the content of which is hereby incorporated by reference in its entirety.

A differentially expressed gene or differential gene expression refer to a gene whose expression is activated to a higher or lower level in a subject suffering from a disorder, such as infertility, relative to its expression in a normal or control subject. The terms also include genes whose expression is activated to a higher or lower level at different stages of the same disorder. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disorder, such as infertility, or between various stages of the same disorder. Differential expression includes both quantitative, as well as qualitative, differences in the temporal or cellular expression pattern in a gene or its expression products. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

In certain embodiments, reverse transcriptase PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure.

The first step is the isolation of mRNA from a target sample. The starting material is typically total RNA isolated from human tissues or fluids.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). The contents of each of theses references is incorporated by reference herein in their entirety. In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE Complete DNA and RNA Purification Kit (EPICENTRE, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In certain embodiments, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Cₜ).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin. For performing analysis on pre-implantation embryos and oocytes, Chuk is a gene that is used for normalization.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, in which internal competitor for each target sequence is used for normalization, and with quantitative comparative PCR using a normalization gene contained within the sample, or a housekeeping gene for RT-PCR. For further details see, e.g. Held et al., Genome Research 6:986 994 (1996), the contents of which are incorporated by reference herein in their entirety.

In another embodiment, a MassARRAY-based gene expression profiling method is used to measure gene expression. In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059 3064 (2003).

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in Yeatman et al. (U.S. patent application number 2006/0195269), the content of which is incorporated by reference herein in its entirety.

In a specific embodiment of the microarray technique, PCR amplified inserts of cDNA clones are applied to a substrate in a dense array, for example, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106 149 (1996), the contents of which are incorporated by reference herein in their entirety). Microarray analysis can be performed by commercially available equipment, following manufacturer's protocols, such as by using the Affymetrix GenChip technology, or Incyte's microarray technology.

Alternatively, protein levels can be determined by constructing an antibody microarray in which binding sites comprise immobilized, preferably monoclonal, antibodies specific to a plurality of protein species encoded by the cell genome. Preferably, antibodies are present for a substantial fraction of the proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, levels of transcripts of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

In other embodiments, Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484 487 (1995); and Velculescu et al., Cell 88:243 51 (1997, the contents of each of which are incorporated by reference herein in their entirety).

In other embodiments Massively Parallel Signature Sequencing (MPSS) is used to measure gene expression. This method, described by Brenner et al., Nature Biotechnology 18:630 634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Immunohistochemistry methods are also suitable for detecting the expression levels of the gene products of the present invention. Thus, antibodies (monoclonal or polyclonal) or antisera, such as polyclonal antisera, specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

In certain embodiments, a proteomics approach is used to measure gene expression. A proteome refers to the totality of the proteins present in a sample (e.g. tissue, organism, or cell culture) at a certain point of time. Proteomics includes, among other things, study of the global changes of protein expression in a sample (also referred to as expression proteomics). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See for example U.S. Pat. Nos. 6,925,389; 6,989,100; and 6,890,763 for further guidance, each of which is incorporated by reference herein in their entirety.

### Microarrays

In certain aspects, the invention provides a microarray including a plurality of oligonucleotides attached to a substrate at discrete addressable positions, in which at least one of the oligonucleotides hybridizes to a portion of a genetic region from Table 1 that includes an infertility-associated variant.

Methods of constructing microarrays are known in the art. See for example Yeatman et al. (U.S. patent application number 2006/0195269), the content of which is hereby incorporated by reference in its entirety.

Microarrays are prepared by selecting probes that include a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods of the invention are preferably immobilized to a solid support, which may be either porous or non-porous. For example, the probes of the invention may be polynucleotide sequences, which are attached to a nitrocellulose or nylon membrane or filter covalently at either the 3′ or the 5′ end of the polynucleotide. Such hybridization probes are well known in the art (see, e.g., Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, the solid support or surface may be a glass or plastic surface. In a particularly preferred embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In preferred embodiments, a microarray comprises a support or surface with an ordered array of binding (e.g., hybridization) sites or “probes” each representing one of the genes described herein, particularly the genes described in Table 1. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to the invention, the microarray is an array (i.e., a matrix) in which each position represents one of the biomarkers described herein. For example, each position can contain a DNA or DNA analogue based on genomic DNA to which a particular RNA or cDNA transcribed from that genetic marker can specifically hybridize. The DNA or DNA analogue can be, e.g., a synthetic oligomer or a gene fragment. In one embodiment, probes representing each of the markers is present on the array. In a preferred embodiment, the array comprises probes for each of the genes listed in Table 1.

As noted above, the probe to which a particular polynucleotide molecule specifically hybridizes according to the invention contains a complementary genomic polynucleotide sequence. The probes of the microarray preferably consist of nucleotide sequences of no more than 1,000 nucleotides. In some embodiments, the probes of the array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR PROTOCOLS: A GUIDE TO METHODS AND APPLICATIONS, Academic Press Inc., San Diego, Calif. (1990). It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083).

Probes are preferably selected using an algorithm that takes into account binding energies, base composition, sequence complexity, cross-hybridization binding energies, and secondary structure. See Friend et al., International Patent Publication WO 01/05935, published Jan. 25, 2001; Hughes et al., Nat. Biotech. 19:342-7 (2001).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93:10539-11286 (1995)).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., MOLECULAR CLONING—A LABORATORY MANUAL (2ND ED.), Vols. 1-3, Cold Spring Harbor Laboratory, Cold Spring Harbor, N.Y. (1989)) could be used. However, as will be recognized by those skilled in the art, very small arrays will frequently be preferred because hybridization volumes will be smaller.

In one embodiment, the arrays of the present invention are prepared by synthesizing polynucleotide probes on a support. In such an embodiment, polynucleotide probes are attached to the support covalently at either the 3′ or the 5′ end of the polynucleotide.

In a particularly preferred embodiment, microarrays of the invention are manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells, which define the locations of the array elements (i.e., the different probes). Microarrays manufactured by this ink-jet method are typically of high density, preferably having a density of at least about 2,500 different probes per 1 cm.sup.2. The polynucleotide probes are attached to the support covalently at either the 3′ or the 5′ end of the polynucleotide.

The polynucleotide molecules which may be analyzed by the present invention are DNA, RNA, or protein. The target polynucleotides are detectably labeled at one or more nucleotides. Any method known in the art may be used to detectably label the target polynucleotides. Preferably, this labeling incorporates the label uniformly along the length of the DNA or RNA, and more preferably, the labeling is carried out at a high degree of efficiency.

In a preferred embodiment, the detectable label is a luminescent label. For example, fluorescent labels, bioluminescent labels, chemiluminescent labels, and colorimetric labels may be used in the present invention. In a highly preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, target polynucleotide molecules from a patient sample are labeled differentially from target polynucleotide molecules of a reference sample. The reference can comprise target polynucleotide molecules from normal tissue samples.

Nucleic acid hybridization and wash conditions are chosen so that the target polynucleotide molecules specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING—A LABORATORY MANUAL (2ND ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, HYBRIDIZATION WITH NUCLEIC ACID PROBES, Elsevier Science Publishers B.V.; and Kricka, 1992, NONISOTOPIC DNA PROBE TECHNIQUES, Academic Press, San Diego, Calif.

Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labeled genetic regions or products of these genetic regions are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor mRNA abundance levels at a large number of sites simultaneously.

### Data Analysis

Method of logistic regression are described, for example in, Ruczinski (Journal of Computational and Graphical Statistics 12:475-512, 2003); Agresti (An Introduction to Categorical Data Analysis, John Wiley & Sons, Inc., 1996, New York, Chapter 8); and Yeatman et al. (U.S. patent application number 2006/0195269), the content of each of which is hereby incorporated by reference in its entirety.

Other algorithms for analyzing associations are known. For example, the stochastic gradient boosting is used to generate multiple additive regression tree (MART) models to predict a range of outcome probabilities. Each tree is a recursive graph of decisions the possible consequences of which partition patient parameters; each node represents a question (e.g., is the FSH level greater than x?) and the branch taken from that node represents the decision made (e.g. yes or no). The choice of question corresponding to each node is automated. A MART model is the weighted sum of iteratively produced regression trees. At each iteration, a regression tree is fitted according to a criterion in which the samples more involved in the prediction error are given priority. This tree is added to the existing trees, the prediction error is recalculated, and the cycle continues, leading to a progressive refinement of the prediction. The strengths of this method include analysis of many variables without knowledge of their complex interactions beforehand.

A different approach called the generalized linear model, expresses the outcome as a weighted sum of functions of the predictor variables. The weights are calculated based on least squares or Bayesian methods to minimize the prediction error on the training set. A predictor's weight reveals the effect of changing that predictor, while holding the others constant, on the outcome. In cases where one or more predictors are highly correlated, in a phenomenon known as collinearity, the relative values of their weights are less meaningful; steps must be taken to remove that collinearity, such as by excluding the nearly redundant variables from the model. Thus, when properly interpreted, the weights express the relative importance of the predictors. Less general formulations of the generalized linear model include linear regression, multiple regression, and multifactor logistic regression models, and are highly used in the medical community as clinical predictors.

Yet another approach that could integrate multiple biomarkers including genetic variants, would be risk score. A risk score is a statistical model that includes and weighs different factors correlated with the phenotype to estimate the disease risk of an individual.

In order to determine expression levels associated with time-to-conception that are statistically significant, a series of logistic regression models may be used. The p-values and odds ratio can be used for statistical inference. Logistic regression models are common statistical classification models. The expression patterns that are statistically significant are considered biomarkers or signatures for the disease.

According to aspects of the invention, the reference TTC signatures can then be used to identify a patient's TTC signatures, classify the patient's time to conception phenotype and tailor treatment of the same.

### Computer Systems

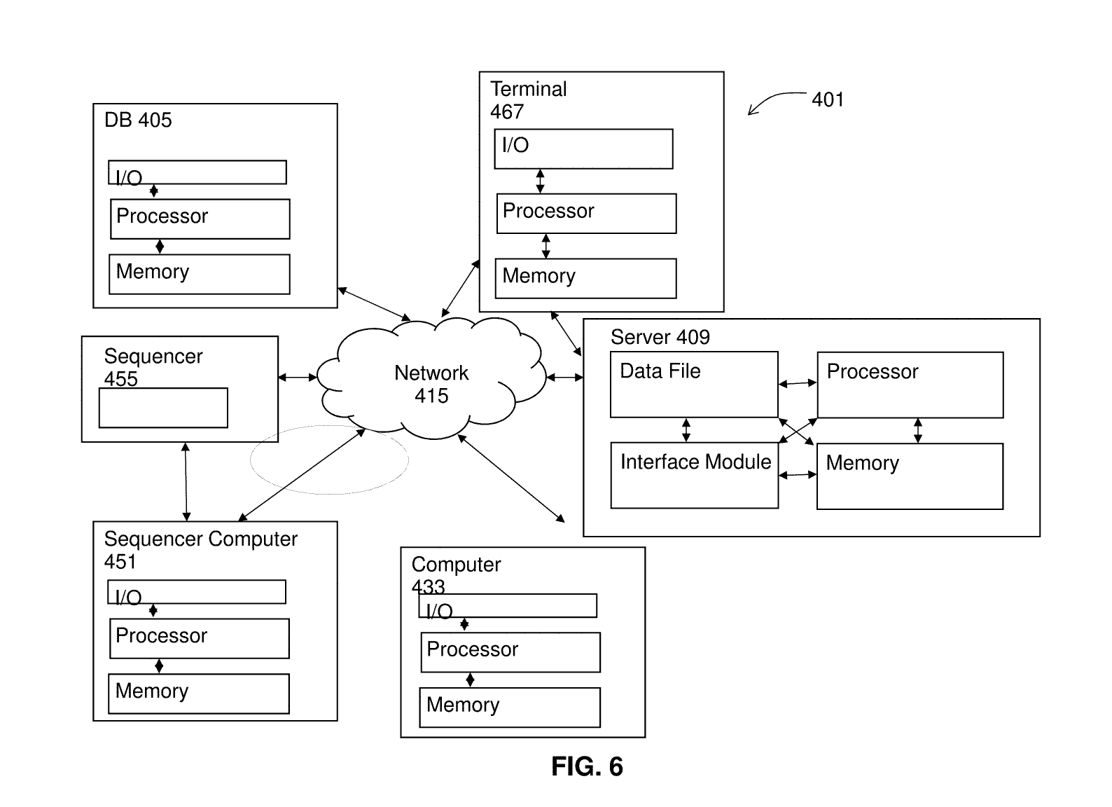

FIG. 6 illustrates a computer system 401 useful for implementing methodologies described herein. A system of the invention may include any one or any number of the components shown in FIG. 6. Generally, a system 401 may include a computer 433 and a server computer 409 capable of communication with one another over network 415. Additionally, data may optionally be obtained from a database 405 (e.g., local or remote). In some embodiments, systems include an instrument 455 for obtaining sequencing data, which may be coupled to a sequencer computer 451 for initial processing of sequence reads.

In some embodiments, methods are performed by parallel processing and server 409 includes a plurality of processors with a parallel architecture, i.e., a distributed network of processors and storage capable of collecting, filtering, processing, analyzing, ranking genetic data obtained through methods of the invention. The system may include a plurality of processors configured to, for example, 1) collect genetic data from different modalities: a) one or more infertility databases 405 (e.g. infertility databases, including private and public fertility-related data), b) from one or more sequencers 455 or sequencing computers 451, c) from mouse modeling, etc; 2) filter the genetic data to identify genetic variations; 3) associate genetic variations with infertility using methods described throughout the application (e.g., filtering, clustering, etc.); 4) determine statistical significance of genetic variations based on fertility criteria defined herein (e.g., Example below); and 5) characterize/identify the genetic variations as infertility biomarkers.

By leveraging genetic data sets obtained across different sources, applying layers of analyses (i.e., filtering, clustering, etc.) to genetic data, and quantifying/qualifying statistical significance of that genetic data, systems of the invention are able yield and identify new infertility biomarkers that previously could not be determined to have any association with infertility. For example, methods of the invention utilize data sets from different modalities. The data sets range include data obtained from infertility databases (e.g., public and private), sequencing data (e.g., whole genome sequencing from one or more biological samples), and genetic data obtained from mouse modeling, etc. Several layers of analysis are then applied to the genetic data to identify whether variations are potentially associated with infertility. Particularly, the genetic data sets are subject to evolutionary conservation analysis, filtering analysis and/or subject to clustering analysis. After those analyses are applied, the variants potentially associated with infertility are then assessed for biological and statistical significance. The variants that are determined to be statistically significant are then classified as infertility biomarkers, even if those variant had no prior association with infertility. Accordingly, using the invention's multi-modal and layered analysis, one is able to identify infertility biomarkers that would not have been identified or associated with infertility using standard techniques (i.e. comparing genetic sequences of an abnormal, infertile population to genetic sequences of a normal, fertile population).

While other hybrid configurations are possible, the main memory in a parallel computer is typically either shared between all processing elements in a single address space, or distributed, i.e., each processing element has its own local address space. (Distributed memory refers to the fact that the memory is logically distributed, but often implies that it is physically distributed as well.) Distributed shared memory and memory virtualization combine the two approaches, where the processing element has its own local memory and access to the memory on non-local processors. Accesses to local memory are typically faster than accesses to non-local memory.

Computer architectures in which each element of main memory can be accessed with equal latency and bandwidth are known as Uniform Memory Access (UMA) systems. Typically, that can be achieved only by a shared memory system, in which the memory is not physically distributed. A system that does not have this property is known as a Non-Uniform Memory Access (NUMA) architecture. Distributed memory systems have non-uniform memory access.

Processor-processor and processor-memory communication can be implemented in hardware in several ways, including via shared (either multiported or multiplexed) memory, a crossbar switch, a shared bus or an interconnect network of a myriad of topologies including star, ring, tree, hypercube, fat hypercube (a hypercube with more than one processor at a node), or n-dimensional mesh.

Parallel computers based on interconnected networks must incorporate routing to enable the passing of messages between nodes that are not directly connected. The medium used for communication between the processors is likely to be hierarchical in large multiprocessor machines. Such resources are commercially available for purchase for dedicated use, or these resources can be accessed via “the cloud,” e.g., Amazon Cloud Computing.

A computer generally includes a processor coupled to a memory and an input-output (I/O) mechanism via a bus. Memory can include RAM or ROM and preferably includes at least one tangible, non-transitory medium storing instructions executable to cause the system to perform functions described herein. As one skilled in the art would recognize as necessary or best-suited for performance of the methods of the invention, systems of the invention include one or more processors (e.g., a central processing unit (CPU), a graphics processing unit (GPU), etc.), computer-readable storage devices (e.g., main memory, static memory, etc.), or combinations thereof which communicate with each other via a bus.

A processor may be any suitable processor known in the art, such as the processor sold under the trademark XEON E7 by Intel (Santa Clara, Calif.) or the processor sold under the trademark OPTERON 6200 by AMD (Sunnyvale, Calif.).

Input/output devices according to the invention may include a video display unit (e.g., a liquid crystal display (LCD) or a cathode ray tube (CRT) monitor), an alphanumeric input device (e.g., a keyboard), a cursor control device (e.g., a mouse or trackpad), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

Any and all references and citations to other documents, such as patents, patent applications, patent publications, journals, books, papers, and web contents, which have been made throughout this disclosure, are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein.

### EXAMPLE

On average, couples of reproductive age in the United States achieve pregnancy within 6 months of starting timed intercourse, but ˜15% take longer than a year or are unable to conceive. Maternal age is often cited as a major determinant of time to conception; however, other clinical or subclinical factors may also contribute. Uncovering these factors, especially in idiopathic cases, will help drive better reproductive care and outcomes. Therefore, a genome-wide association study (GWAS) for time-to-first-conception phenotype was conducted in more than 36,000 women. The objective of the GWAS was to identify genetic variants associated with a time to conception greater than or equal to 13 months for first pregnancy among women 35 years old or younger. The GWAS resulted in discovery of two newly identified loci with statistically significant correlation with time-to-first-conception.

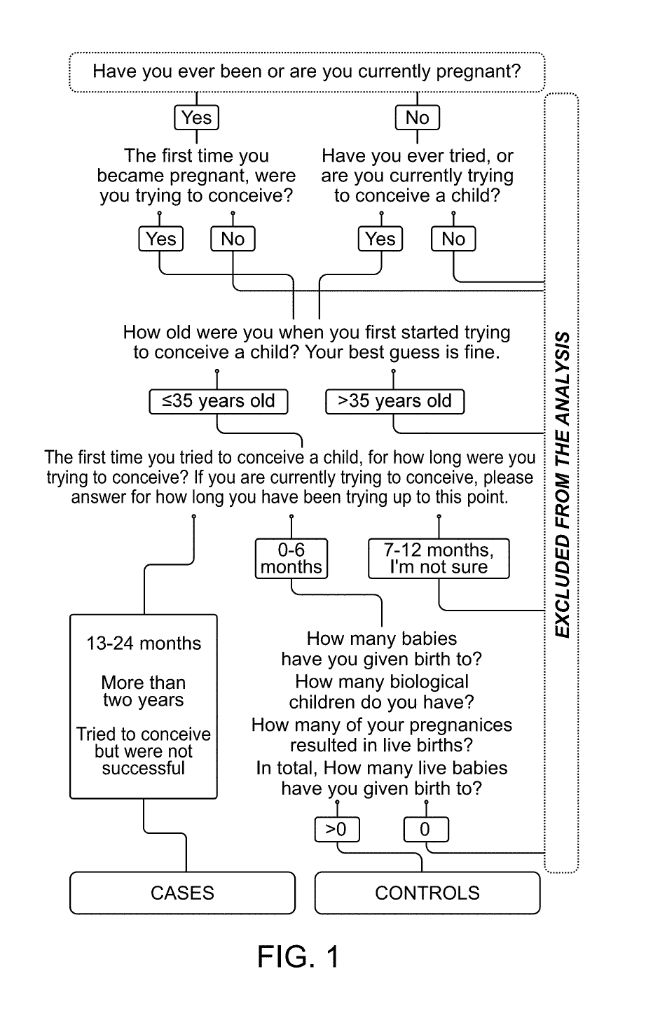

The GWAS was performed on 36k women of European ancestry. Cases were women who reported trying to conceive for ≥13 months in their first pregnancy attempt (n=9,822). Controls were women with ≥1 biological child who reported trying to conceive for <6 months in their first pregnancy attempt (n=26,947). A flowchart of the case-control ascertainment process is shown in FIG. 1. Samples were genotyped on 1 of 4 versions of a custom, genome-wide genotyping array targeting between 556 and 955K genetic variants. Samples were then imputed for 15M variants using phase I of the 1000 Genomes Project as a reference. Assuming an additive model, we tested logistic regression on the time-to-first-conception phenotype, including age, first 5 principal components, and genotyping array version as covariates. Association results were adjusted for the observed genomic control inflation factor of the p-values distribution (λ=1.019). We developed and utilized an in-house database to annotate the reproductive biological functions of the proximal genes within the statistically significant loci and to identify previously reported associations between the variants in linkage disequilibrium with the index variant and reproductive traits.

Results

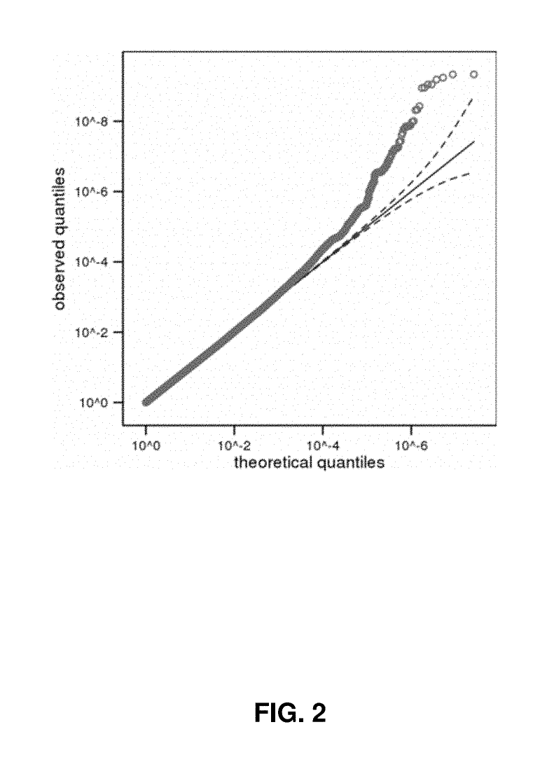

Among women who reported trying to conceive their first child at ≤35 years old, 67.9% reported a time to conception of <6 months and 20.1% reported a time to conception of ≥13 months. The top of the quantile-quantile curve deviates from the null hypothesis (FIG. 2), suggesting a genetic component for our time to first conception phenotype. FIG. 2 shows a quantile-quantile plot of association results from the GWAS on time to first conception of ≥13 months. The distribution of the ˜15 million p-values generated in our GWAS (circles) is compared to the distribution of the p-values expected under the null hypothesis (line). The lower half of the distribution follows the null distribution, indicating no inflation. The upper half of the distribution deviates from the null, indicating an enrichment of low p-values.

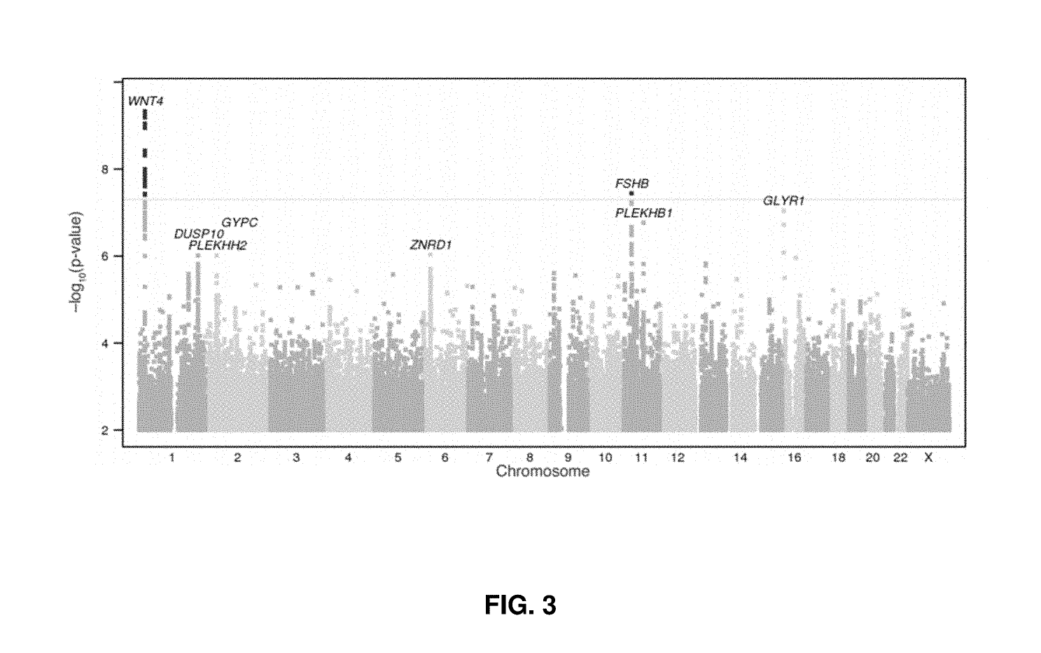

Two genomic loci reached genome-wide significance (p<5×10⁻⁸) in association with time to first conception of ≥13 months. FIG. 3 shows a Manhattan plot of these data, showing the two loci, covering the WNT4 and FSHB genes, reaching genome-wide significance. Table 3, below, shows the top genomic loci (p<1×10⁻⁶) associated with the phenotype.

| Variant | | Position in | Nearest | Risk | Other | Risk allele | Odds ratio | |
|---|---|---|---|---|---|---|---|---|
| ID | Chromosome | GRCh37 | gene(s) | allele | allele | frequency | [95% Cl] | P-value |
| rs61768001 | 1 | 22465820 | WNT4 | C | T | 0.15 | 1.16 | 4.6 × 10⁻¹⁰ |
| | | | | | | | [1.11, 1.22] | |
| rs11031006 | 11 | 30226528 | FSHB | A | G | 0.14 | 1.14 | 3.6 × 10⁻⁸ |
| | | | | | | | [1.33, 1.39] | |
| rs78061108 | 16 | 4875454 | GLYR1 | A | C | 0.01 | 1.58 | 9.1 × 10⁻⁸ |
| | | | | | | | [1.33, 1.82] | |
| rs1278297 | 11 | 7334487 | FAM168A | C | T | 0.97 | 1.30 | 1.7 × 10⁻⁷ |
| | | | PLEKHB1 | | | | [1.18, 1.44] | |
| rs79875368 | 2 | 126942380 | GYPC | T | C | 0.01 | 1.60 | 6.8 × 10⁻⁷ |
| | | | | | | | [1.34, 1.92] | |
| rs1150743 | 6 | 29992261 | HLA-A | T | C | 0.11 | 1.14 | 9.2 × 10⁻⁷ |
| | | | ZNRD1 | | | | [1.08, 1.20] | |
| rs112671841 | 1 | 222296708 | DUSP10 | Ins | Del | 0.34 | 1.09 | 9.6 × 10⁻⁷ |
| | | | HHIPL2 | | | | [1.06, 1.13] | |
| rs13013678 | 2 | 43854836 | THADA | G | A | 0.58 | 1.09 | 9.6 × 10⁻⁷ |
| | | | PLEKHH2 | | | | [1.05, 1.13] | |

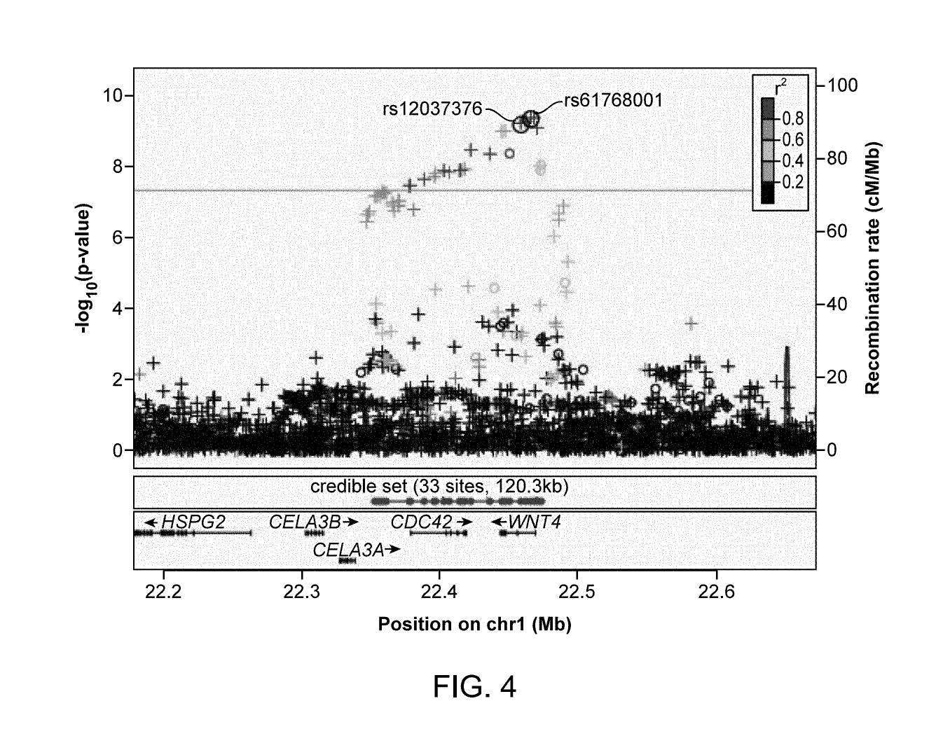

The rs61768001 single nucleotide polymorphism (SNP) in the WNT4 gene was the most strongly associated SNP in one of these two loci (p=4.6×10⁻¹⁰, OR=1.16). FIG. 4 c. The color of each associated variant represents a measure of the linkage disequilibrium (LD) with the most strongly associated SNP for this locus (rs61768001). It had an overall minor allele frequency of 15% in our dataset. WNT4 has been linked to regulation of both pre- and postnatal uterine development and function. For example, rare missense variants within WNT4 have been found in patients with Müllerian aplasia. WNT4 may also regulate human endometrial decidualization, and deletion of Wnt4 in the mouse uterus compromises embryo implantation and causes subfertility. The WNT4 locus has also been associated with endometriosis in published GWAS. In fact, rs12037376, the top SNP of the WNT4 locus identified in a recently published endometriosis GWAS, is in linkage disequilibrium (LD) with the SNP associated with the strongest signal in our time-to-first conception GWAS, suggesting that the signals in these GWASs could be generated by the same functional SNP (FIG. 4).

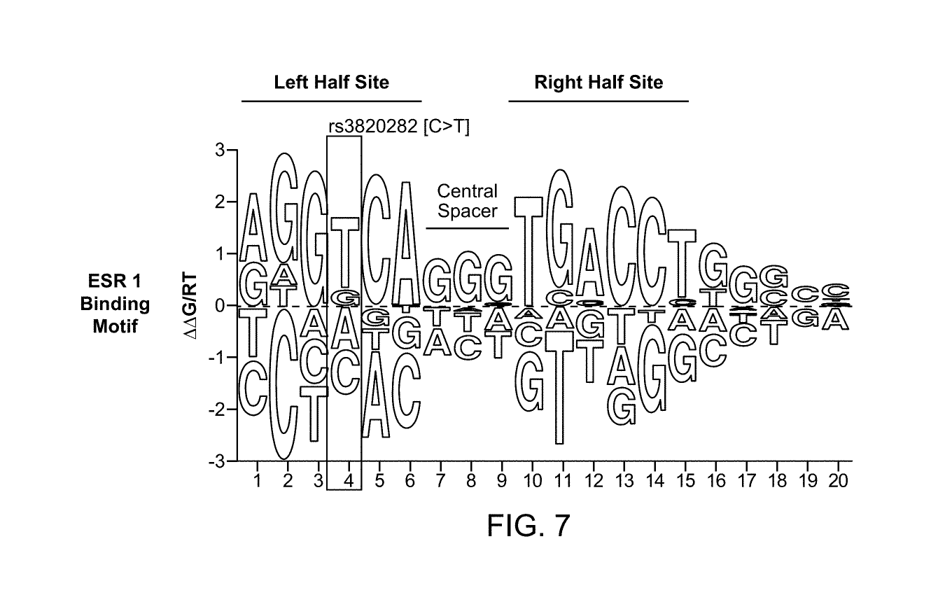

FIG. 7 depicts the location of the variant rs3820282 of WNT4, which is in high linkage disequilibrium with rs61768001. rs3820282 is located within the left half-site of one of the two predicted estrogen response elements (ERE) in intron 1 of WNT4. The variant rs3820282 modulates the binding of Estrogen Receptor 1 (ESR1) in the WNT4 locus, with the allele associated with higher time-to-conception increasing the binding of ESR1. Estrogen regulates endometrium receptivity and reduced or increased estrogen levels can impair the ability of the embryo to implant.

An increased binding of ESR1 to WNT4, make WNT4 more responsive to estrogen, and impact the regulation of embryo implantation. Overexpression of WNT4 in human endometrial stromal cells (HESCs) markedly advanced the differentiation program and resulted in a great increase in expression of the decidualization markers IGFBP1 and PRL. An increased ESR1 binding of WNT4 could make WNT4 more responsive to estrogen, potentially advancing decidualization and jeopardizing the synchronicity between the decidualization and ovulation.

The C allele of rs61768001 and the T allele of rs3820282, associate with longer time to first conception. Administration of certain drugs, for example, estrogen may improve or reduce the time-to-conception in the presence of these biomarkers. For example, the presence of he allele of the variant associated with a higher response to estrogen in WNT4 indicates that a lower dose of estrogen should be administered as part of the embryo transfer or alone. The invention includes calculating the dosage of a fertility treatment based on the presence of certain alleles identified to improve time-to-conception and/or a woman's fertility. The dose, or course of treatment, may be calculated based on the presence of the allele alone, or the calculation may include other factors, including but not limited to age, BMI, diagnosis of other disease or infertility related conditions.

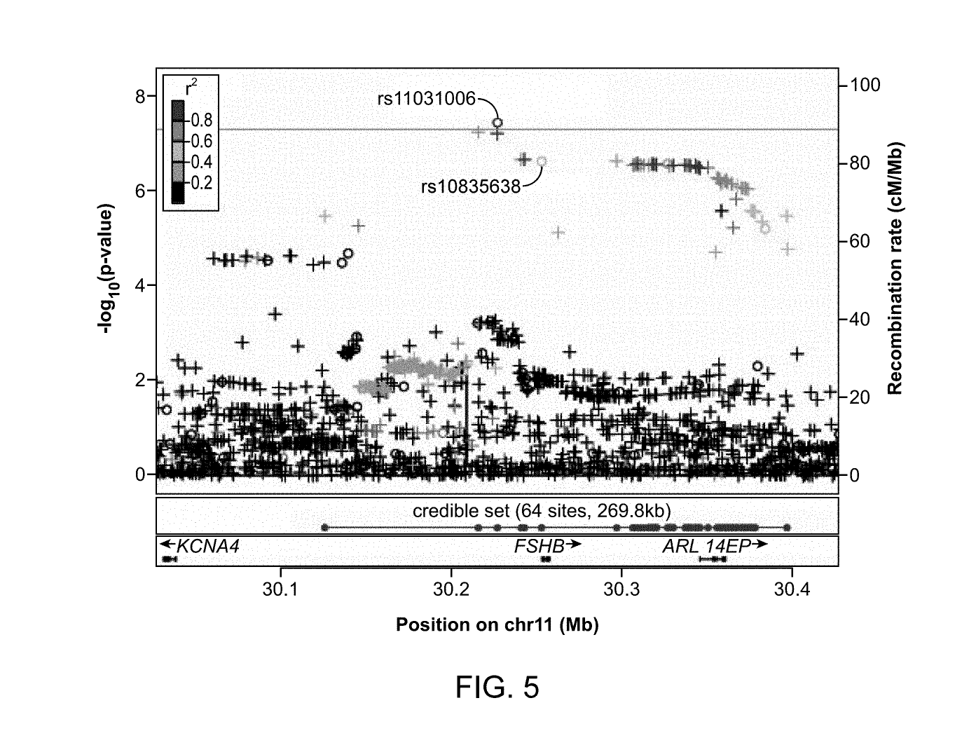

The most strongly associated variant in the second of our two loci with genome-wide significance, rs11031006 (p=3.6×10⁻⁸, OR=1.14), is ˜25 kb upstream of FSHB, which encodes the β-subunit of follicle stimulating hormone (FSH) FIG. 5 shows a regional association plot of the 11p14.1 locus, which includes the FSHB gene. The color of each associated variant represents a measure of the linkage disequilibrium with the most strongly associated SNP for this locus (rs11031006). It had an overall minor allele frequency of 14% in our dataset.

Interestingly, another variant in the FSHB promoter (rs10835638), which is in LD with the top SNP that we detected (rs11031006) (FIG. 5), has been associated with reduced FSH serum levels, polycystic ovary syndrome (PCOS), longer menstrual cycles, increased age of menopause, and higher rates of female nulliparity. The rs11031006-A allele, associated with first conception in ≥13 months according to our analysis, has also been associated in the literature with later age at menarche, first live birth, and menopause, but also lower dizygotic twinning rates and lower lifetime parity. The rs10835638-T allele (commonly referred to as-211G>T) reduces the binding affinity of the transcription factor LIM Homeobox 3 (LHX3) and reduces FSHB transcription in gonadotrope cells. In women, this lowered FSH level could impact the process of oocytes maturation and increase time-to-conception as observed in our study. These associations from the literature are shown in Table 4 below. The A and T alleles of rs11031006 and rs10835638 respectively, associate with longer time to first conception in our analysis, and have been associated with other female reproductive traits in the literature.

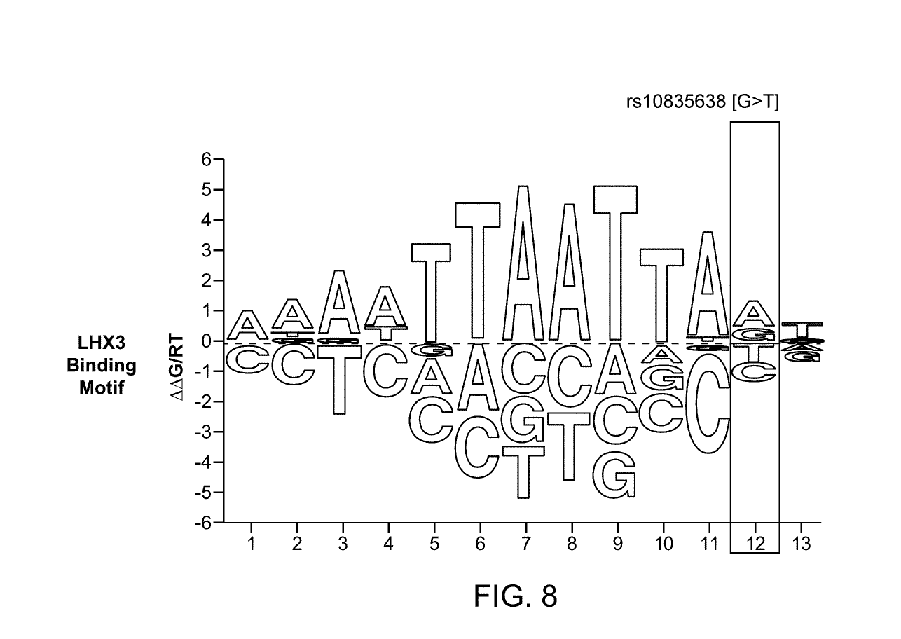

FIG. 8 depicts the location of the variant rs10835638, and is located in the FSHB promoter, within an 11-bp binding site of the homeodomain transcription factor LHX3.

| Associated with decreased . . . | Associated with increased . . . |
|---|---|
| Serum FSH levels | Duration of menstrual cycles |
| Lifetime parity | Age of menarche |
| Twinning rates | Age of menopause |
| Endometriosis risk | Age of first birth |
| | Incidence of female nulliparity |
| | PCOS risk |

Only information on first conception/birth was available for our study participants. Therefore, we were unable to assess whether trends in time to first conception were present in subsequent pregnancies.

Some pregnancies occurred years before participants completed the survey for our study. Although this time lapse may introduce a risk of recall bias, a recent study suggests that recall of time to pregnancy, even after 24-28 years, closely reflects time to pregnancy recorded prospectively. Moreover, this study also suggested that dichotomous measures of time to pregnancy (e.g. ‘more than’ or ‘less than’ 13 cycles) are well reported by study participants. Importantly, questions used to identify cases and controls were phrased in this way in our study.

Our analysis did not account for medical conditions or lifestyle factors that could affect times to conception, such as endometriosis or smoking. Our results suggest that variants in genes involved in uterine development, endometrial function, and/or gonadotropin signaling potentially influence the time to first conception. To our knowledge, this study is the first to evaluate genetic determinants of time to conception. It includes a large sample size of women from the general population in contrast to other reproductive health studies that may only focus on women diagnosed with infertility. Further investigation will reveal the specific biological processes responsible for the phenotype.

1. A method for assessing an increased time-to-conception phenotype in a female, the method comprising:
obtaining a sample from a female subject;
analyzing the sample to determine a presence of a biomarker associated with endometriosis, uterine development, endometrial function, or gonadotropin signaling; and
determining a probability of increased time-to-conception based on the presence of the biomarker.

2. The method of claim 1, wherein the biomarker is a gene.

3. The method of claim 2, wherein analyzing comprises sequencing a portion of the gene to determine a presence or absence of a variant.

4. The method of claim 3, wherein the variant comprises a single nucleotide polymorphism, a deletion, an insertion, a rearrangement, a copy number variation, or a combination thereof

5. The method of claim 1, wherein the biomarker is associated with the WNT4 or FSHB genes.

6. The method of claim 1, wherein the biomarker is a gene product.

7. The method of claim 6, wherein the gene product comprises RNA or protein.

8. The method of claim 1, wherein the sample comprises human tissue or body fluid.

9. The method of claim 8, wherein the sample comprises blood.

10. The method of claim 1, wherein the sample comprises nucleic acid of the female subject.

11. The method of claim 1, further comprising the step of determining course of treatment based on the probability of increased time-to-conception.

12. The method of claim 11, wherein the treatment is selected from the group consisting of: in vitro fertilization, fertility drugs, and intrauterine insemination.

13. A method for identifying genetic factors associated with infertility in a female, the method comprising:
obtaining sample genetic data from a plurality of females suspected of infertility;
genotyping the sample genetic data on a genome-wide array;
conducting a regression to correlate regions of the sample genetic data with an infertility trait; and
identifying a locus among the regions that has a statistically significant correlation with the infertility trait.

14. The method of claim 13, wherein the sample comprises human tissue or body fluid .

15. The method of claim 13, wherein the genome-wide array comprises biomarkers associated with endometriosis, uterine development, endometrial function, or gonadotropin signaling.

16. The method of claim 15, wherein the biomarkers comprise WNT4 and FSHB.

17. The method of claim 13, wherein the infertility trait comprises a reported time to first conception of greater than twelve months.

18. An array for assessing a time to conception phenotype, the array comprising:
a substrate; and
a plurality of oligonucleotides attached to the substrate at discrete addressable positions, wherein at least one of the oligonucleotides hybridizes to a portion of a gene selected from the group including WNT4 and FSHB.

19. A method for determining a course of treatment to decrease time-to-conception, the method comprises:
obtaining a sample from a female subject;
analyzing the sample to determine presence of a biomarker associated with increased time-to-conception, wherein the biomarker is associated with endometriosis, uterine development, endometrial function, or gonadotropin signaling; and
determining course of treatment based on the presence of the biomarker.

20. The method of claim 19, wherein the determining step comprises identifying a dose of fertility drugs based on the presence of the biomarker.
